(19) United States
(12) Patent Application Publication
(10) Pub. No.: US 2024/0226280 A1
(43) Pub. Date: Jul. 11, 2024

(54) IMMUOGENIC COMPOSITIONS OF MUTANT SARS-COV-2 N PROTEIN AND GENE AND METHODS OF USE THEREOF

(71) Applicant: King Abdullah University of Science and Technology, Thuwal (SA)

(72) Inventors: Muhammad Shuaib, Thuwal (SA); Tobias Mourier, Thuwal (SA); Arnab Pain, Thuwal (SA)

(21) Appl. No.: 18/559,272

(22) PCT Filed: May 4, 2022
(86) PCT No.: PCT/IB2022/054126

§ 371 Date: Nov. 6, 2023

Related U.S. Application Data
(60) Provisional application No. 63/183,933, filed on May 4, 2021.

(51) Int. Cl. A61K 39/215; A61K 39/00

(52) U.S. Cl. CPC A61K 39/215; A61K 2039/5256; A61K 2039/53; A61K 2039/575

(57) ABSTRACT

Compositions and methods for generating an immune response to fight against viral infections such as SARS-CoV-2 are provided. The disclosed compositions and methods are based on the discovery of the three consecutive SNPs (G28881A, G28882A, G28883C) underlying the R203K/G204R mutation in the SARS-CoV-2 N protein, associated with increased immune system response when expressed in cells. The immune responses that can be upregulated by the disclosed compositions include antibody production and/or upregulation of immune related genes that are generally involved in host defense against viral and bacterial infections, for example, increased expression of one or more genes including, but not limited to SHFL, MX1, AMD9L, TRIM22, TRIM14, EIF2 AK2, etc.

The compositions include a peptide of the SARS-CoV-2 N-protein including the R203K/G204R mutation, a fragment thereof, or a nucleic acid encoding the same.

The compositions are administered to a subject in need thereof to elicit an immune response in the subject.

### CROSS REFERENCE TO RELATEDS APPLICATIONS

The present application claims priority to U.S. Application No. 63/183,933, filed May 4, 2021 the disclosure of which is incorporated herein by reference in its entirety.

### FIELD OF THE INVENTION

This invention is generally in the field of compositions and methods for eliciting an immune response in a subject in need thereof, against pathogens such as viruses, for example coronaviruses, and particularly, SARS-CoV-2.

### BACKGROUND OF THE INVENTION

The emergence of novel severe acute respiratory syndrome coronavirus 2 (SARS-CoV-2), which causes the respiratory coronavirus infectious disease 2019 (COVID-19), resulted in a pandemic that has triggered an unparalleled public health emergency^{1,2}. The global spread of SARS-CoV-2 depended fundamentally on human mobility patterns. The vast majority of vaccines currently in clinical development are based only on the spike protein, or parts thereof, and seem to protect against disease but not against infection. A mystery surrounding the COVID-19 pandemic has been the relatively low case numbers and especially deaths, in sub-Saharan Africa compared to other regions worldwide. Underreporting of cases due to insufficient testing is a likely factor in the lower COVID-19 numbers reported in sub-Saharan Africa. However, a major excess mortality beyond that expected for the region has not been observed between March 2020 and August 2021, arguing against large numbers of missed cases. Studies have unveiled the presence of cross-reactive antibodies to SARS-CoV-2 in pre COVID-19 blood samples from subjects in these populations. Borrega, et al., Viruses, 13(11):2325 (2021). Thus compositions that can elicit a broad immune responses are useful in scenarios involving pathogenic infections such as that encountered with virus, and particularly, SARS-CoV-2.

It is an object of the present invention to provide compositions for immune responses in a subject in need thereof.

It is also an object of the present invention to provide methods eliciting an immune response in a subject.

### SUMMARY OF THE INVENTION

Compositions and methods for generating an immune response to fight against viral infections such as SARS-CoV-2 are provided. The disclosed compositions and methods are based on the discovery of the three consecutive SNPs (G28881A, G28882A, G28883C) underlying the R203K/G204R mutation in the SARS-CoV-2 N- protein relative to the N-protein of the Wuhan isolate identified within NCBI Reference Sequence: NC_045512.2, i.e., NCBI Reference Sequence: YP_009724397.2 (SEQ ID NO: 1), associated increased expression of immune-related processes when transfected into cells. The immune responses that can be upregulated by the disclosed compositions include antibody production in response to the protein or a translated nucleic acid encoding the mutant N protein or a fragment thereof, and/or upregulation of immune related genes that are generally involved in host defense against viral and bacterial infections, for example, increased expression of one or more genes including, but not limited to SHFL, MX1, AMD9L, TRIM22, TRIM14, EIF2AK2, etc. The compositions include a peptide fragment of the SARS-CoV-2 N- protein including the R203K/G204R mutation or a nucleic acid encoding the same. The nucleic acid is preferably, mRNA. The mRNA-based compositions encode the antigen of interest, herein a fragment of the SARS-CoV-2 N-protein comprising the R203K/G204R and contain 5′ and 3′ untranslated regions (UTRs), a 5′ cap and a poly(A) tail, and in optional embodiments for self-amplifying RNAs the viral replication machinery that enables intracellular RNA amplification and abundant protein expression. The mRNA composition in some preferred embodiments, is delivered to a subject in need thereof, using nanoparticles, for example, lipid nanoparticles. In some embodiments the compositions include an adjuvant.

The compositions are administered to a subject in need thereof to elicit an immune response in the subject. In one embodiment, the immune response is against SARS-CoV-2.

### BRIEF DESCRIPTION OF THE FIGURES

FIG. 1 shows the 836 detected SNPs are shown along their positions in the SARS-CoV-2 genome (x-axis) and their frequency in the Saudi samples (y-axis). High-frequency SNPs are highlighted along with the 3 SNPs underlying the R203K/G204R changes in the N protein (G28881A;G2882A;G28883C) (tope panel); bottom panel: Scatter plot of SNP frequencies in Saudi samples (y-axis) and in global, non-Saudi samples available from GISAID in 2020. SNPs differing by at least 0.1 in absolute values are highlighted in blue.

FIG. 2A. Top: The numbers of samples from Saudi Arabia presented in this study are shown as bars by their sampling date (January 2020-March 2021). Bottom: Samples deposited in GISAID. On both plots, lines show the fraction of samples having the R203K/G204R SNPs (red line), having both the R203K/G204R SNPs and the Spike protein N501Y SNP (blue line), and having the Spike protein D614G SNP (green line). FIG. 2B. Overview of the three SNPs underlying the N protein R203K/G204R changes. Amino acid numbers in the N protein are shown above. FIG. 2C. Boxplot showing the distribution of virus copy number derived from Ct measurements. Ct values from the N1 primer pairs were normalized by RNase P primer pair values and converted to copy numbers from a standard curve. Only samples processed using the TaqPath™ kit (Thermofisher) were included (see Supplementary Information). Copy numbers are shown for four different haplotypes (as indicated below the plot) corresponding to virus genome positions 28,881-28,883 (orange text) and 23,403 (blue text). ‘Wuhan’ denotes the genotypes in the reference genome (NC_045512). FIG. 2D. Manhattan plot showing the association between SARS-CoV-2 SNPs and recorded mortality in our samples set. Negative log₁₀(uncorrected p-values) from Fisher's exact tests are shown as red circles. Gene boundaries are indicated by background colors (listed on top), and the three R203K/G204R SNPs (positions 28,881-28,883) in the N gene are highlighted. FIG. 2E. Global samples from GISAID (Feb. 24, 2021).

Left: Counts of partial and full R203K/G204R SNPs at genome position 28,881-28,883, where the Wuhan reference has the GGG genotype. Right: Counts of R203K/G204R SNPs in different Nextstrain clades. FIGS. 2F-G. Co-occurrences of SNPs shown as Jaccard Index (F) and log2 odds-ratio (G). The co-occurrence between the three SNPs in the R203K and G204R mutations (genomic mutations shown above plots) and all SNPs present in at least 20 samples (x-axes) are shown as circles. Co-occurrences between the three SNPs are highlighted in orange.

FIGS. 3A-G. Show RNA binding and Affinity Purification Mass-Spectrometry (AP-MS) analysis of mutant and control SARS-CoV-2 N protein. FIG. 3A is a schematic diagram showing the SARS-CoV-2 N protein different domains (Upper: control, Lower: mutant) and highlighting the mutation site (R203K and G204R) and the linker region (LKR) containing a serine-arginine rich motif (SR-motif). The bar-plot (lower panel) indicates the SIFT²⁹ predicted deleteriousness score of substitution at position 204 from G to R. FIG. 3B is a sketch of In-vitro RNA immunoprecipitation (RIP) procedure used for analysis of viral RNA interaction with mutant and control N protein (See methods for details). Isolated RNAs were analyzed by RT-qPCR using specific primers for viral N gene (N1 and N2) and E gene. FIG. 3C is a bar chart showing the level of viral RNA retrieval (% input) with mutant and control N protein (+SD from n=3 independent experiments, [two-sided t-test, p-values N1:0.00080 (***), N2:0.00088 (***), E:0.008 (**), S:0.00059 (***), and ORFlab:0.002 (**)]). FIG. 3D. Identification of host-interacting partners of mutant and control SARS-CoV-2 N protein by Affinity Mass-Spectrometry. Heatmap showing significantly differentially changed human proteins (3 replicates) interactome in mutant versus control N protein AP-MS analysis. FIG. 3E. Gene Ontology (GO)-enrichment analysis of significantly changed terms between mutant and control proteins in terms of biological process and pathway enrichment. The scale shows p-value adjusted Log2 of odds ration mutant-versus-control. FIG. 3F. Profiling of phosphorylation status of mutant and control N protein by Mass-Spectrometry. Sketch showing part of SR-rich motif of SARS-CoV-2 N protein containing the KR mutation site (R203K and G204R) (Lower). The hyper-phosphorylated serine 206 (as shown in FIG. 3G) in the mutant N protein near the KR mutation site is indicated with the triangle. FIG. 3G.

Phosphorylation status of mutant and control N protein was analyzed by mass spectrometry (±SD from n=3 biologically independent experiments per affinity condition). Bar-plot shows the Log2 intensities of phosphorylated peptide (Serine 206) in control and mutant condition.

FIGS. 4A-4D show Transcriptional profiling of mutant and control N transfected cells. HEK293T cells were transfected with plasmids expressing the full-length N-control and N-mutant protein along with mock control. 48-hour post-transfection total RNA was isolated and subjected to RNA-sequencing using illumina NovaSeq 6000 platform. FIG. 4A. Heatmap shows normalized expression of top significantly differentially expressed genes in N-mutant and N-control conditions (adj p-value<0.05 and log2 fold-change cutoff≥1). Genes enriched in interferon and immune related processes are overexpressed in the N-mutant transfected cells. The heatmap was generated by the visualization module in the NetworkAnalyst. FIG. 4B. Plot showing comparison of fold-changes for up-regulated genes in N-mutant and N-control conditions. Differentially expressed genes display higher up-regulation in the N-mutant condition (as orange dots that represent common up-regulated genes are skewed towards the lower half of the diagonal). FIG. 4C. Venn diagram shows the common and unique up-regulated genes in both conditions. FIG. 4D. GO-enrichment analysis of uniquely up-regulated genes in the N-mutant condition. The enriched GO BP(Biological Processes) term is related to interferon response. The enriched terms display an interconnected network with overlapping gene sets (from the list). Each node represents an enriched term and colored by its p-value (red shows smallest p-value). The size of each node corresponds to number of linked genes from the list.

FIG. 5A. For the 20 SNPs showing the highest levels of within-host polymorphisms (see Supplementary Information), the number of samples with polymorphisms of this SNP (y-axes) and the number of samples with that SNP in the assembled reference genomes (x-axes) was plotted for each hospital. Each circle therefore represents a hospital. The correlation between these two parameters were then calculated (table, right). Four SNPs had polymorphisms but were not present in any assembled genome, and correlation could not be calculated (‘NA’ in table). Plots are shown for the five SNPs with the highest instances of polymorphisms as well as one of the SNPs (G28882A) from the R203K/G204R mutations. FIG. 5B. Bar chart showing the number and collection dates of samples from King Abdullah Medical Centre in Jeddah. The number of samples from deceased patients are shown as black squares on the bars, and the number of samples containing the R203K/G204R SNPs shown as open circles. FIGS. 5C-D. Oligomerization analysis of mutant and control N protein. FIGS. 5C. BS³ cross-linking (2 mM) and SDS-PAGE analysis of the oligomerization forms of mutant and control N proteins. FIG. 5D. Densitometry analysis of bands corresponding to oligomeric forms (trimer and tetramer) was performed. Bar-plot represents the relative intensities from three independent experiments (as shown mean±SD). (t-test, p value (0.000203***) and (0.00427**).

FIGS. 6A-6E. Affinity mass spectrometry (AP-MS) analysis of mutant and control SARS-CoV-2 N protein and host protein interaction. FIG. 6A. Sketch showing the workflow of affinity mass spectrometry procedure. HEK-293 cell expressing 2XStrep-tagged control and mutant N protein were used for MagStrep affinity purification. Purified proteins were separated on SDS-PAGE and subjected to silver staining and western blotting for confirmation. After confirmation, interacting proteins were analyzed by mass spectrometry. FIG. 6B. (Upper) Silver staining of control and mutant N protein associated host proteins (1 and 2 show two loading volume). (Lower) Western blot confirmation of N protein (mutant and control) using anti-Strep antibody. FIG. 6C. Correlation matrix of three replicates for control and mutant N protein AP-MS. FIG. 6D. Overlapping of identified N interacting proteins with N-interacting proteins reported in previous study²⁷ (Gordon et al., 2020 Nature). FIG. 6E. Volcano plot displaying the differential interactions of pairwise comparisons (mutant_vs_control) in—Log10 adj. p-values vs. the Log2 protein fold change. Proteins with statistically significant (Adjusted p-value<=0.05, and Log fold change>=1) difference between mutant and control AP-MS conditions are highlighted.

FIGS. 7A-E. Transcriptomic analysis of mutant and control N transfected host cells. FIG. 7A. PCA on transcriptome of HEK293T cells transfected with plasmids expressing the full-length N-control and N-mutant protein along with mock control. FIGS. 7B-C. Volcano-plot showing differentially expressed (DE) genes based on a filtering criterion of adj p-value<0.05 and fold-change cutoff≥1) as determined by the method EdgeR in NetworkAnalyst tool. X-axis depicts log2 fold-change of DE genes and Y-axis depicts —log10 P-value. Genes with significant up-regulation are shown in red and down-regulated are shown in blue. All other non-significant genes are shown in gray. FIG. 7D. Plot showing the distribution of log 2-fold changes in both N-mutant and N-control conditions. FIG. 7E. GO enrichment analysis of all up-regulated genes in the N-mutant condition. The enriched GO BP(Biological Processes) terms are displayed by plotting against the—log10 of the false discovery rate (FDR q value). The enriched terms display an interconnected network with overlapping gene sets (from the list). Each node represents an enriched term and colored by its FDR q value (as shown in the bar-chart). The size of each node corresponds to number of linked genes from the list.

### DETAILED DESCRIPTION OF THE INVENTION

Compositions and methods for generating an immune response to pathogens such as viruses, particularly, SARS-CoV-2 are provided. The disclosed compositions and methods are based on the discovery of the three consecutive SNPs (G28881A, G28882A, G28883C) underlying the R203K/G204R mutation in the SARS-CoV-2 Nucleocapsid (N) protein, associated with higher viral loads in COVID-19 patients and that cells and cells transfected to express this mutant form showed significant up regulation of genes involved in immune-related processes. The compositions include a peptide fragment of the SARS-CoV-2 N- protein or a fragment thereof, including the R203K/G204R mutation or a nucleic acid encoding the same. In one embodiment, the peptide is a full length mutant N-protein, however, the peptide can be a fragment thereof.

The nucleic acid is preferably, mRNA. The mRNA composition in some preferred embodiments, is delivered to a subject in need thereof, using nanoparticles, for example, lipid nanoparticles. In some embodiments the compositions include an adjuvant.

The compositions are administered to a subject in need thereof to elicit immune-related responses, including but not limited to antibody production against SARS-CoV-2 and upregulation of immune related genes/processes.

### I. DEFINITIONS

As used herein, the term “adjuvant” refers to a compound or mixture that enhances an immune response.

As used herein, the term “effective amount” or “therapeutically effective amount” means a dosage sufficient to treat, inhibit, or alleviate one or more symptoms of a disease state being treated or to otherwise provide a desired pharmacologic effect. The precise dosage will vary according to a variety of factors such as subject-dependent variables (e.g., age, immune system health, etc.), the disease, and the age of the subject.

As used herein, the term “gene” refers to a nucleic acid (e.g., DNA or RNA) sequence that including coding sequences necessary for the production of a polypeptide, RNA (e.g., including, but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation.

As used herein “immunogenic composition” means that the composition can induce an immune response. By “immune response” means any reaction by the immune system. These reactions include the alteration in the activity of an organism's immune system in response to an antigen and can involve, for example, antibody production, induction of cell-mediated immunity, complement activation, development of immunological tolerance or upregulation of immune-related genes.

As used herein, “oral,” “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a vaccine composition from the mouth, administration of a vaccine composition through a nasojejunal or gastrostomy tube, intraduodenal administration of a vaccine composition, and rectal administration.

As used herein, “mammal” includes both humans and non-humans and include but is not limited to humans, non-human primates, canines, felines, murines, bovines, equines, and porcines.

As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “host cell” refers to a cell into which a recombinant vector can be introduced.

As used herein, “transform” and “transfect” encompass the introduction of a nucleic acid (e.g. a vector) into a cell by a number of techniques known in the art.

### II. COMPOSITION

The disclosed compositions include peptides or nucleic acid molecules, specifically polynucleotides, primary constructs and/or mRNA which encode a fragment of the SARS-CoV-2 N protein including the R203K/G204R mutation. The nucleic acid or peptide is included in a formulation suitable for administration to a subject in need thereof.

### A. Nucleic Acids Encoding the N Protein

Disclosed compositions can include nucleic acids encoding mutant N proteins as disclosed herein, preferably, in a vector for delivery and expression in cells, preferably mammalian cells. The disclosed compositions and methods are based on the discovery of the three consecutive SNPs (G28881A, G28882A, G28883C) relative to Wuhan isolate identified within NCBI Reference Sequence: NC_045512.2. The CDS for N protein from NC_045512 is reproduced below, with the sequences that are mutated identified in capital letters and underlined.

(SEQ ID NO: 37)

| | |
|---|---|
| 28261 | atgtctg ataatggacc ccaaaatcag |
| | cgaaatgcac cccgcattac |
| 28321 | gtttggtgga ccctcagatt caactggcag taaccagaat |
| | ggagaacgca gtggggcgcg |
| 28381 | atcaaaacaa cgtcggcccc aaggtttacc caataatact |
| | gcgtcttggt tcaccgctct |
| 28441 | cactcaacat ggcaaggaag accttaaatt ccctcgagga |
| | caaggcgttc caattaacac |
| 28501 | caatagcagt ccagatgacc aaattggcta ctaccgaaga |
| | gctaccagac gaattcgtgg |
| 28561 | tggtgacggt aaaatgaaag atctcagtcc aagatggtat |
| | ttctactacc taggaactgg |
| 28621 | gccagaagct ggacttccct atggtgctaa caaagacggc |
| | atcatatggg ttgcaactga |
| 28681 | gggagccttg aatacaccaa aagatcacat tggcacccgc |
| | aatcctgcta acaatgctgc |
| 28741 | aatcgtgcta caacttcctc aaggaacaac attgccaaaa |
| | ggcttctacg cagaagggag |
| 28801 | cagaggcggc agtcaagcct cttctcgttc ctcatcacgt |
| | agtcgcaaca gttcaagaaa |
| 28861 | ttcaactcca ggcagcagta GGGgaacttc tcctgctaga |
| | atggctggca atggcggtga |
| 28921 | tgctgctctt gctttgctgc tgcttgacag attgaaccag |
| | cttgagagca aaatgtctgg |
| 28981 | taaaggccaa caacaacaag gccaaactgt cactaagaaa |
| | tctgctgctg aggcttctaa |
| 29041 | gaagcctcgg caaaaacgta ctgccactaa agcatacaat |
| | gtaacacaag ctttcggcag |
| 29101 | acgtggtcca gaacaaaccc aaggaaattt tggggaccag |
| | gaactaatca gacaaggaac |
| 29161 | tgattacaaa cattggccgc aaattgcaca atttgccccc |
| | agcgcttcag cgttcttcgg |
| 29221 | aatgtcgcgc attggcatgg aagtcacacc ttcgggaacg |
| | tggttgacct acacaggtgc |
| 29281 | catcaaattg gatgacaaag atccaaattt caaagatcaa |
| | gtcattttgc tgaataagca |
| 29341 | tattgacgca tacaaaacat tcccaccaac agagcctaaa |
| | aaggacaaaa agaagaaggc |
| 29401 | tgatgaaact caagccttac cgcagagaca gaagaaacag |
| | caaactgtga ctcttcttcc |
| 29461 | tgctgcagat ttggatgatt tctccaaaca attgcaacaa |
| | tccatgagca gtgctgactc |
| 29521 | aactcaggcc taa. |

Therefore, the coding sequence for full length mutant N protein can be represented at least by the sequence:

(SEQ ID NO: 38)

atgtctg ataatggacc ccaaaatcag
cgaaatgcac cccgcattac gtttggtgga ccctcagatt
caactggcag taaccagaat ggagaacgca gtggggcgcg
atcaaaacaa cgtcggcccc aaggtttacc caataatact
gcgtcttggt tcaccgctct cactcaacat ggcaaggaag
accttaaatt ccctcgagga caaggcgttc caattaacac
caatagcagt ccagatgacc aaattggcta ctaccgaaga
gctaccagac gaattcgtgg tggtgacggt aaaatgaaag
atctcagtcc aagatggtat ttctactacc taggaactgg
gccagaagct ggacttccct atggtgctaa caaagacggc
atcatatggg ttgcaactga gggagccttg aatacaccaa
aagatcacat tggcacccgc aatcctgcta acaatgctgc
aatcgtgcta caacttcctc aaggaacaac attgccaaaa
ggcttctacg cagaagggag cagaggcggc agtcaagcct
cttctcgttc ctcatcacgt agtcgcaaca gttcaagaaa
ttcaactcca ggcagcagta AACgaacttc tcctgctaga
atggctggca atggcggtga tgctgctctt gctttgctgc
tgcttgacag attgaaccag cttgagagca aaatgtctgg
taaaggccaa caacaacaag gccaaactgt cactaagaaa
tctgctgctg aggcttctaa gaagcctcgg caaaaacgta
ctgccactaa agcatacaat gtaacacaag ctttcggcag
acgtggtcca gaacaaaccc aaggaaattt tggggaccag
gaactaatca gacaaggaac tgattacaaa cattggccgc
aaattgcaca atttgccccc agcgcttcag cgttcttcgg
aatgtcgcgc attggcatgg aagtcacacc ttcgggaacg
tggttgacct acacaggtgc catcaaattg gatgacaaag
atccaaattt caaagatcaa gtcattttgc tgaataagca
tattgacgca tacaaaacat tcccaccaac agagcctaaa
aaggacaaaa agaagaaggc tgatgaaact caagccttac
cgcagagaca gaagaaacag caaactgtga ctcttcttcc
tgctgcagat ttggatgatt tctccaaaca attgcaacaa
tccatgagca gtgctgactc aactcaggcc taa;

the SNP in mutant N protein are identified in capital letters and underlined.

Thus, in one embodiment, the nucleic acid encoding mutant N protein is SEQ ID NO:38 or a fragment thereof, up to a 50, 60, 70, 80, 90%, 91%, 92%, 93%, 94%, 95%, 96%, 97%, 98%, or 99% identical to SEQ ID NO:38.

In preferred embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. The mRNA in some aspects is encoded by SEQ ID NO:38 or a fragment thereof, up to a 50%, 60%, 70%, 80%, 90%, 91%, 92%, 93%, 94%, 95%, 96%, 97%, 98%, or 99% identical to SEQ ID NO:38. Preferred nucleic acid fragments encode at least 10 amino acids that span the region containing the R203K/G204R mutation for example, SSRGTSPARM (SEQ ID NO. 33) with residues 203 and 204 underlined, containing the R203K/G204R mutation i.e., SSKRTSPARM (SEQ ID NO: 34) with the mutations underlined, STPGSSKRTS; (SEQ ID NO: 35), SKRTSPARMA (SEQ ID NO: 36), etc.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Hamann, et al., J. Biol. Eng., 13:7 (2019) demonstrated that gene expression in hBMSCs driven by cytomegalovirus (CMV) promoter, resulted in 10-fold higher transgene expression than transfection with plasmids containing elongation factor 1 α (EF1α) or rous sarcoma virus (RSV) promoters.

Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

Recent transfection studies have investigated minicircle DNA (mcDNA), nucleic acids that are derived from pDNA by recombination that removes bacterial sequences. L1 RNA can be introduced into host cells using mcDNA using methods known in the art (Mun et al. Biomaterials, 2016; 101:310-320).

The vectors including the nucleic acid of interest can be administered to subjects in need thereof resulting in transfection or transformation of the cells in the subject which in turn express the protein/peptide encoded by the nucleic acid.

### B. Mutant N— Protein

The N protein of SARS-CoV-2, an abundant viral protein within infected cells, serves multiple functions during viral infection, which besides RNA binding, oligomerization, and genome packaging, playing essential roles in viral transcription, replication, and translation^{30,51} Also, the N protein can evade immune response and perturbs other host cellular processes such as translation, cell cycle, TGFβ signaling, and induction of apoptosis⁵² to enhance virus survival. The critical functional regulatory hub within the N protein is a conserved serine-arginine (SR) rich-linker region (LKR), which is involved in RNA and protein binding⁵³, oligomerization^{33,34}, and phospho-regulation^{35,40}.

Mutant N proteins useful in the disclosed compositions and methods have R203K/G204R mutation in the SARS-CoV-2 N-protein, when compared to the N-protein of the Wuhan isolate identified by NCBI Reference Sequence: YP_009724397.2,

(SEQ ID NO: 1)

msdngpqnqr napritfggp sdstgsnqng ersgarskqr
rpqglpnnta swftaltqhg kedlkfprgq gvpintnssp
ddqigyyrra trrirggdgk mkdlsprwyf yylgtgpeag
lpygankdgi iwvategaln tpkdhigtrn pannaaivlq
lpqgttlpkg fyaegsrggs qassrsssrs rnssrnstpg
ssRGtsparm agnggdaala lllldrinql eskmsgkgqq
qqgqtvtkks aaeaskkprq krtatkaynv tqafgrrgpe
qtqgnfgdqe lirqgtdykh wpqiaqfaps asaffgmsri
gmevtpsgtw ltytgaikld dkdpnfkdqv illnkhiday
ktfpptepkk dkkkkadetq alpqrqkkqq tvtllpaadl
ddfskqlqqs mssadstqa

in which the residues that are mutated within mutant N protein, capitalized and underlined. Thus, an exemplary full length mutant N protein is:

(SEQ ID NO: 39)

msdngpqnqr napritfggp sdstgsnqng ersgarskqr
rpqglpnnta swftaltqhg kedlkfprgq gvpintnssp
ddqigyyrra trrirggdgk mkdlsprwyf yylgtgpeag
lpygankdgi iwvategaln tpkdhigtrn pannaaivlq
lpqgttlpkg fyaegsrggs qassrsssrs rnssrnstpg
ssKRtsparm agnggdaala lllldrinql eskmsgkgqq
qqgqtvtkks aaeaskkprq krtatkaynv tqafgrrgpe
qtqgnfgdqe lirqgtdykh wpqiaqfaps asaffgmsri
gmevtpsgtw ltytgaikld dkdpnfkdqv illnkhiday
ktfpptepkk dkkkkadetq alpqrqkkqq tvtllpaadl
ddfskqlqqs mssadstqa.

The protein can be a full length mutant N protein, or a fragment thereof. Preferably, the fragment should include at least 10 amino acids that span the region containing the R203K/G204R mutation for example, SSRGTSPARM (SEQ ID NO. 33) with residues 203 and 204 underlined, containing the R203K/G204R mutation i.e., SSKRTSPARM (SEQ ID NO: 34) with the mutations underlined, STPGSSKRTS; (SEQ ID NO: 35), SKRTSPARMA (SEQ ID NO:36), etc. Thus the mutant N protein can be up to a 50, 60, 70, 80, 90%, 91%, 92%, 93%, 94%, 95%, 96%, 97%, 98%, or 99% identical to the amino acid sequence of SEQ ID NO: 1, and additionally includes the R203K/G204R mutation. Thus, a fragment of the mutant N protein disclosed herein is suitably at least 10 amino acids in length, suitably at least 25 amino acids, suitably at least 50 amino acids, suitably at least 100 amino acids, suitably at least 200 amino acids etc., suitably the majority of the polypeptide of interest. Suitably a fragment comprises a whole motif or a whole domain of SEQ ID NO:38.

The disclosed mutant N proteins/peptides are incorporated into pharmaceutical formulations as disclosed herein, for administration to a subject in need thereof, to elicit an immune response.

Sequence Identity homology

Sequence homology can be considered in terms of functional similarity (i.e., amino acid residues or nucleotide codons having similar chemical properties/functions), and it preferably, is express homology in terms of sequence identity. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). One of ordinary skill in the art is aware of conservative substitutions which can be made to an amino acid sequence with the expectation of retaining function. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu).

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids). Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalizing unduly the overall homology (identity) score. This is achieved by inserting “gaps” in the sequence alignment to try to maximize local homology/identity.

These more complex methods assign “gap penalties” to each gap that occurs in the alignment so that, for the same number of identical amino acids, a sequence alignment with as few gaps as possible-reflecting higher relatedness between the two compared sequences-will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is—12 for a gap and—4 for each extension. Calculation of maximum percent homology therefore firstly requires the production of an optimal alignment, taking into consideration gap penalties. A suitable computer program for conducting such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

### C. Adjuvants

The compositions include one or more adjuvants. Exemplary adjuvants include, but are not limited to, aluminum hydroxide, aluminum phosphate, he emulsion adjuvants, MF59 (5% Squalene, 0.5% Tween 80, and 0.5% Span 85, formulated into submicron particles using a microfluidizer), and AS03. LR agonists have been extensively studied as vaccine adjuvants. CpG, Poly I:C, glucopyranosyl lipid A (GLA), and resiquimod (R848) are agonists for TLR9, TLR3, TLR4, and TLR7/8, respectively.

RNA (e.g., mRNA) vaccines combined with the flagellin adjuvant (e.g., mRNA-encoded flagellin adjuvant) have been shown to have superior properties in that they may produce much larger antibody titers and produce responses earlier than commercially available vaccine formulations. While not wishing to be bound by theory, it is believed that the RNA (e.g., mRNA) vaccines, for example, as mRNA polynucleotides, are better designed to produce the appropriate protein conformation upon translation, for both the antigen and the adjuvant, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Useful adjuvants but are not limited to, one or more set forth below: Mineral Containing Adjuvant Compositions include mineral salts, such as aluminum salts and calcium salts. Exemplary mineral salts include hydroxides (e.g., oxyhydroxides), phosphates (e.g., hydroxyphosphates, orthophosphates), sulfates, and the like or mixtures of different mineral compounds (e.g., a mixture of a phosphate and a hydroxide adjuvant, optionally with an excess of the phosphate), with the compounds taking any suitable form (e.g., gel, crystalline, amorphous, and the like), and with adsorption to the salt(s) being preferred. The mineral containing compositions can also be formulated as a particle of metal salt (WO/0023105). Aluminum salts can be included in compositions of the invention such that the dose of A13+ is between 0.2 and 1.0 mg per dose.

Additional adjuvants for use in the compositions are submicron oil-in-water emulsions. Examples of submicron oil-in-water emulsions include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s-—n-glycero-3-huydroxyphosphophoryloxy)-ethylamine (MTP-PE), for example, the MF59 (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes.

Bioadhesives and mucoadhesives can also be used as adjuvants in the invention. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). In some aspects, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19: 109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s--n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27: 571-577, 2002 and Jones, “Resiquimod 3M”, Curr Opin Investig Drugs 4: 214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

### D. Formulations and Carriers

The composition of the invention can be formulated in pharmaceutical compositions including a pharmaceutically acceptable excipient, carrier, buffer, stabilizer, or other materials well known to those skilled in the art. Such materials should typically be non-toxic and should not typically interfere with the efficacy of the active ingredient. The precise nature of the carrier or other material can depend on the route of administration, e.g., oral, cutaneous or subcutaneous, nasal, intramuscular, or intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose, or other saccharide solution or glycols such as ethylene glycol, propylene glycol, or polyethylene glycol (PEG) can be included. The term “carrier” refers to a diluent, adjuvant, excipient, or vehicle with which the pharmaceutical composition (e.g., immunogenic or vaccine formulation) is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, ethanol and the like. Examples of suitable pharmaceutical carriers are described in “Remington's Pharmaceutical Sciences” by E. W. Martin. The formulation should be selected according to the mode of administration.

For cutaneous, or subcutaneous injection, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required.

Administration is preferably in a “therapeutically effective amount” or “prophylactically effective amount” (as the case can be, although prophylaxis can be considered therapy), this being sufficient to show benefit to the individual.

### III. METHODS OF USE

The disclosed formulations are administered to a subject in need thereof such as a human subject to elicit an immune response in the subject, including but not limited to, antibody production, and/or upregulation of immune related genes in cells in the subject., for example, immune related genes shown in Table 5, which include genes involved in innate response against viral and bacterial infections. Examples include, but are not limited, OAS1 (2-5′-oligoadenylate synthetase 1) encodes a protein that synthesizes 2′,5′-oligoadenylates (2-5As)-this protein activates latent RNase L, which results in viral RNA degradation and the inhibition of viral replication; OAS3 (2′-5′-oligoadenylate synthetase 3) encodes a adsRNA-activated antiviral enzyme which plays a critical role in cellular innate antiviral response (IFN-induced); BST2 (bone marrow stromal cell antigen 2) encodes an IFN-induced antiviral host restriction factor which efficiently blocks the release of diverse mammalian enveloped viruses by directly tethering nascent virions to the membranes of infected cells; DDX60 (DExD/H-box helicase 60) encodes a DEXD/H box RNA helicase that functions as an antiviral factor and promotes RIG-I-like receptor-mediated signaling; DDX58 (DExD/H-box helicase 58) encodes and innate immune receptor that senses cytoplasmic viral nucleic acids and activates a downstream signaling cascade leading to the production of type I interferons and proinflammatory cytokines; RSAD2 (radical S-adenosyl methionine domain containing 2) protein encoded by this gene is an interferon-inducible antiviral protein that belongs to the S-adenosyl-L-methionine (SAM) superfamily of enzymes and plays a role in cellular antiviral response and innate immune signaling. Antiviral effects result from inhibition of viral RNA replication, interference in the secretory pathway, binding to viral proteins and dysregulation of cellular lipid metabolism; EIF2AK2 (eukaryotic translation initiation factor 2 alpha kinase 2), encodes an IFN-induced dsRNA-dependent serine/threonine-protein kinase that phosphorylates the alpha subunit of eukaryotic translation initiation factor 2 (EIF2S1/eIF-2-alpha) and plays a key role in the innate immune response to viral infection; TRIM14 (Tripartite motif-containing 14), protein encoded by this gene is a member of the tripartite motif (TRIM) family. The TRIM motif includes three zinc-binding domains, a RING, a B-box type 1 and a B-box type 2, and a coiled-coil region.-Plays an essential role in the innate immune defense against viruses and bacteria; TRIM22 (tripartite motif containing 22), protein encoded by this gene localizes to the cytoplasm and its expression is induced by interferon and is involved in innate immunity against different DNA and RNA viruses; MX1 (MX Dynamin Like GTPase 1) encodes a guanosine triphosphate (GTP)-metabolizing protein that participates in the cellular antiviral response; SHFL (shiftless Antiviral Inhibitor Of Ribosomal Frameshifting), the encoded protein binds nucleic acids and inhibits programmed-1 ribosomal frameshifting required for translation by many RNA viruses. Viruses inhibited by the protein include Zika virus, dengue virus and the coronaviruses, SARS-CoV and SARS-CoV2.

The subject can be about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger. In some embodiments, the subject is a young adult between the ages of about 20 years and about 50 years (e.g., about 20, 25, 30, 35, 40, 45 or 50 years old). In some embodiments, the subject is an elderly subject about 60 years old, about 70 years old, or older (e.g., about 60, 65, 70, 75, 80, 85 or 90 years old).

In vivo gene therapy can be employed, whereby the genetic material is transferred directly into the patient. In these embodiments, genetic material is introduced into a patient by a virally derived vector or by non-viral techniques. In vivo nucleic acid therapy can be accomplished by direct transfer of a functionally active DNA into mammalian somatic tissue or organ in vivo. Nucleic acids be administered in vivo by viral means. A therapeutic gene expression cassette is typically composed of a promoter that drives gene transcription, the transgene of interest, and a termination signal to end gene transcription. Such an expression cassette can be embedded in a plasmid (circularized, double-stranded DNA molecule) as delivery vehicle. Plasmid DNA (pDNA) can be directly injected in vivo by a variety of injection techniques, among which hydrodynamic injection achieves the highest gene transfer efficiency in major organs by quickly injecting a large volume of pDNA solution and temporarily inducing pores in cell membrane. To help negatively charged pDNA molecules penetrate the hydrophobic cell membranes, chemicals including cationic lipids and cationic polymers have been used to condense pDNA into lipoplexes and polyplexes, respectively.

Nucleic acid molecules encoding the disclosed mutant N protein may be packaged into retrovirus vectors using packaging cell lines that produce replication-defective retroviruses, as is well-known in the art. Other virus vectors may also be used, including recombinant adenoviruses and vaccinia virus, which can be rendered non-replicating. Nucleic acids may also be delivered by other carriers, including liposomes, polymeric micro- and nanoparticles and polycations such as asialoglycoprotein/polylysine. Various techniques and methods for in vivo gene delivery using the disclosed vectors and carriers are known in the art (reviewed in Wang, et al., Discov. Med., 18(97):67-77 (2014). A major advancement in DNA vector design is minicircle DNA (mcDNA), which differs from pDNA in the lack of bacteria-derived, CpG-rich backbone sequences. When administered in vivo, mcDNA mediates safer, higher and more sustainable transgene expression than conventional pDNA.

In some aspects, the disclosed compositions (e.g., LNP-encapsulated mRNA compositions) produce prophylactically- and/or therapeutically efficacious levels, concentrations and/or titers of antigen-specific antibodies or effective upregulation of immune-related genes in cells in the subject to which they are administered.

As defined herein, the term antibody titer refers to the amount of antigen-specific antibody produces in a subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc. In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces>0.5 μg/ml, >0.1 μg/ml, >0.2 μg/ml, >0.35 μg/ml, >0.5 μg/ml, >1 μg/ml, >2.mu.g/ml, >5 μg/ml or >10 μg/ml. In exemplary embodiments of the invention, an efficacious vaccine produces>10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.). In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

Particularly preferred embodiments are exemplified below.

### EXAMPLES

### Methods

### Sample Collection

As part of the study, nasopharyngeal swab samples were collected in 1 ml of TRIzol (Ambion, USA) from 892 COVID-19 patients with various grades of clinical disease manifestations—consisting of severe, mild and asymptomatic symptoms. The anonymized samples were amassed from 8 hospitals and one quarantine hotel located in Madinah, Makkah, Jeddah and Riyadh.

Ethical approval was obtained from the Institutional review board of the Ministry of Health in Makkah region with the numbers H-02-K-076-0420-285 and H-02-K-076-0320-279, as well as the Institutional review board of Dr. Sulaiman Al Habib Hospital number RC20.06.88 for samples from Riyadh and the Eastern regions, respectively.

### RNA Isolation

RNA was extracted using the Direct-Zol RNA Miniprep kit (Zymo Research, USA) following the manufacturer's instructions, along with several optimization steps to improve quality and quantity of RNA from clinical samples. The optimization included extending the TRIzol incubation period, and the addition of chloroform during initial lysis step to obtain the aqueous RNA layer. The quality control of purified RNA was performed using Broad Range Qubit kit (Thermo Fisher, USA) and RNA 6000 Nano LabChip kit (Agilent, USA) respectively. RT-PCR was conducted using the one-step Super Script III with Platinum Taq DNA Polymerase (Thermo Fisher, USA) and TaqPath COVID-19 kit (Applied Biosystems, USA) on the QuantStudio 3 Real-Time PCR instrument (Applied Biosystems, USA) and 7900 HT ABI machine. The primers and probes used were targeting two regions in the nucleocapsid gene (N1 and N2) in the viral genome following the Centre for Disease Control and prevention diagnostic panel, along with primers and probe for human RNase P gene (CDC; fda.gov/media/134922/download). Samples were considered COVID positive once the cycle threshold (Ct) values for both N1 and N2 regions were less than 40. For amplicon seq purposes, the samples chosen were of Ct less than 35 to ensure successful genome assembly in order to upload on GISAID.

### Sequencing and Data Analysis

cDNA and amplicon libraries were prepared using the COVID-19 ARTIC-V3 protocol, producing˜ 400 bp amplicons tiling the viral genome using V3 nCoV-2019 primers (Wellcome Sanger Institute, UK; dx.doi.org/10.17504/protocols.io.beuzjex6). Amplicons were then processed for deep, paired-end sequencing with the Novaseq 6000 platform on the SP 2×250 bp flow cell type (Illumina, USA).

### Genome Assembly, SNP and Indel Calling

Illumina adapters and low-quality sequences were trimmed using Trimmomatic v0.38⁶⁰. Reads were mapped to SARS-CoV-2 Wuhan-Hu-1 NCBI reference sequence NC_045512.2 using BWA⁶¹. Mapped reads were processed using GATK v 4.1.7 pipeline commands MarkDuplicatesSpark, HaplotypeCaller, VariantFiltration, SelectVariants, BaseRecalibrator, ApplyBQSR, and HaplotypeCaller to identify variants⁶² High quality SNPs were filtered using the filter expression:

$$“ QD < 2. ‖ FS > 60. ‖ SOR > 3. ‖ MQRankSum < - 12.5 ‖ ReadPosRankSum < - 8. ”$$

High quality Indels were filtered using the filter expression:

$$“ QD < 2. ‖ FS > 200. ‖ SOR > 10. ‖ ReadPosRankSum < - 20. ”$$

Consensus sequences were generated by applying the good quality variants from GATK on the reference sequence using bcftools consensus command⁶³. Regions which are covered by less than 30 reads are masked in the final assembly with 'N's.

Consensus assembly sequences were deposited to GISAID¹¹. To retrieve high-confidence SNPs assembled sequences were re-aligned against the Wuhan-Hu-1 reference sequence (NC_045512.2), and only positions in the sample sequences with unambiguous bases in a 7-nucleotide window centered around the SNP position were kept for further analysis.

### Phylogenetic Analysis

To generate the phylogeny of Saudi samples with a global context, a total of 308,012 global sequences were downloaded from GISAID on 31 Dec. 2020, filtered and processed using Nextstrain pipeline¹². Global sequences were grouped by country and sample collection month and 20 sequences per group were randomly sampled which resulted in 10,873 global representative sequences and 952 Saudi sequences. The phylogeny was constructed using IQ-TREE⁶⁴, clades were assigned using Nextclade and internal node dates were inferred and sequences pruned using TreeTime⁶⁵. Nextstrain protocol was followed for the above-mentioned steps. The resulting global phylogenetic tree was reduced to retain the branches that lead to Saudi leaf nodes and visualized using baltic library (https://github.com/evogytis/baltic).

### Phylodynamic Analysis

Phylodynamic analyses use the same sequence subset used in the full phylogenetic analysis, extracted from the GISAID SARSCoV-2 database¹¹. Wrapper functions for the importation date estimates and skygrowth model are provided in the sarscov2R package as compute timports' and ‘skygrowth1’ respectively (github.com/emvolz-phylodynamics/sarscov2Rutils).

### Importation Date Estimates for Nextstrain Clades

Sequences corresponding to each Nextstrain¹² clade were extracted using the Nextstrain_clade parameter in the GISAID metadata table. A subset of 500 international sequences were select for each clade based on Tamura Nei 93 distance with tn93 (github.com/veg/tn93) and stratified over time⁶⁶. A maximum likelihood phylogeny with an HKY substitution model for each clade was estimated with IQtree^{64,67}. Time-scaled phylogenies were estimated from this using treedater with a strict molecular clock constrained between 0.0009 and 0.0015 substitutions per site per year⁶⁸. 15 Variations of each dated phylogeny were produced by collapsing small branches and resolving polytomies. The state of each internal node was reconstructed by maximum parsimony with the phangorn R package⁶⁹. Importation events are estimated at the midpoint of a branch along which a location change is inferred to occur by this method.

### Estimation of Donor Countries Behind Importation Events

To identify import events that resulted in new introductions into Saudi Arabia, 25,198 sequences were subsampled from 590K global sequences available on GISAID on February 24th 2021. Samples with closer genetic distance to Saudi Samples were preferred. The phylogeny was constructed using IQ-TREE⁶⁴, internal nodes dates and possible country for internal nodes were inferred using TreeTime⁶⁵. Nextstrain protocol was followed for the above-mentioned steps¹². In house scripts were used to traverse the global phylogenetic tree to identify branches that resulted in transitions into Saudi Arabia from another country.

### Skygrowth Model

Sequences from Saudi Arabia available on GISAID on Dec. 31, 2020 were used to construct effective population size and growth rate of SARS-CoV2 in Saudi Arabia over the course of the first wave of the epidemic (March to September 2020). As with the importation date estimates, a maximum likelihood phylogeny was produced, time-scaled and variation introduced by resolving polytomies to give a sample of 15 phylogenies.

The growth rate and effective population size over time on these phylogenies was modelled using the R package skygrowth¹⁶. Skygrowth is a non-parametric Bayesian approach which applies a stochastic process on estimates of growth rate and effective population size. The model included mean-centered, unit variance estimates of travel rates from google mobility data (google.com/covid19/mobility/) as a covariate (transit stations percent change from baseline), 60 timesteps and a tau (precision) value corresponding to a 1% change in growth per week. The growth rate output was converted to an estimate of R over time using an infectious period of 9.5 days⁷⁰.

### Origin of R203K/G204R SNPs

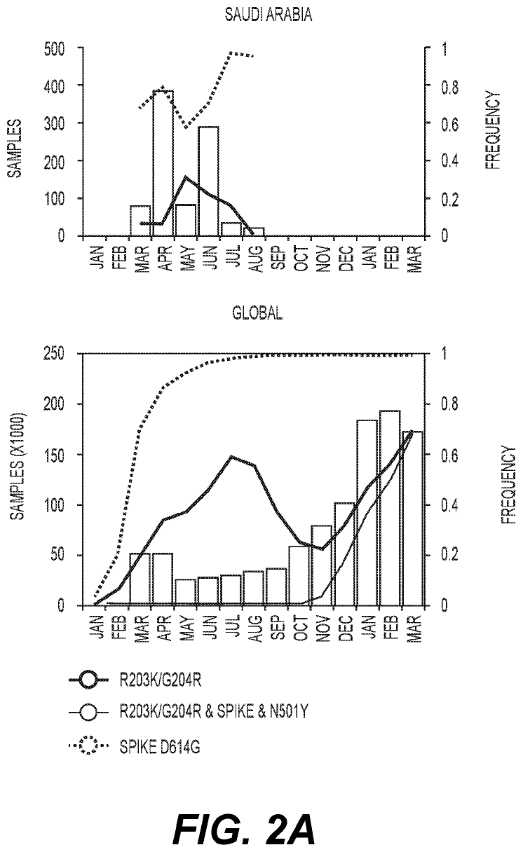

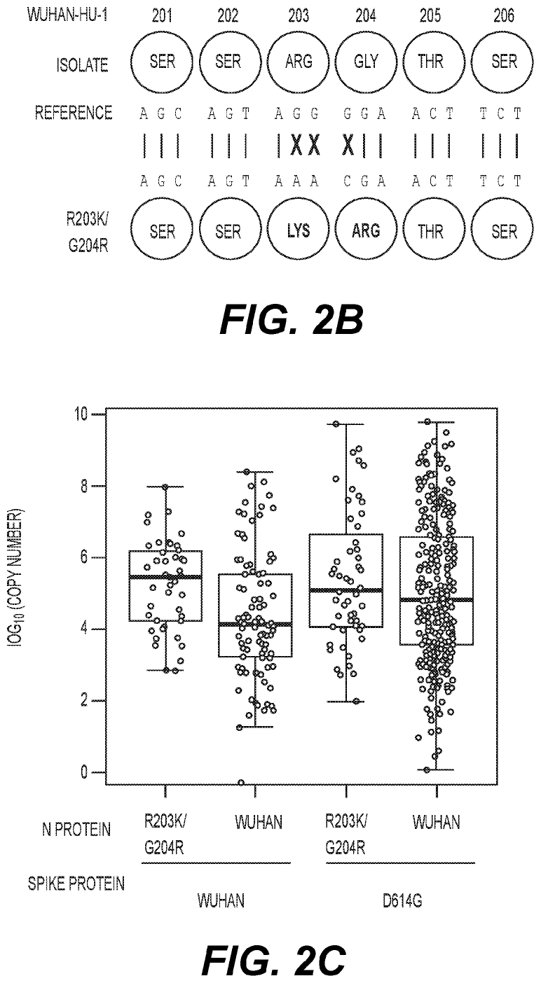

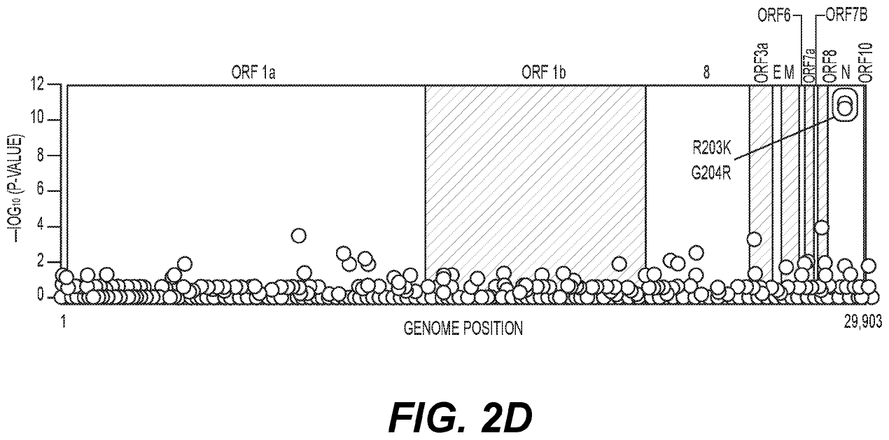

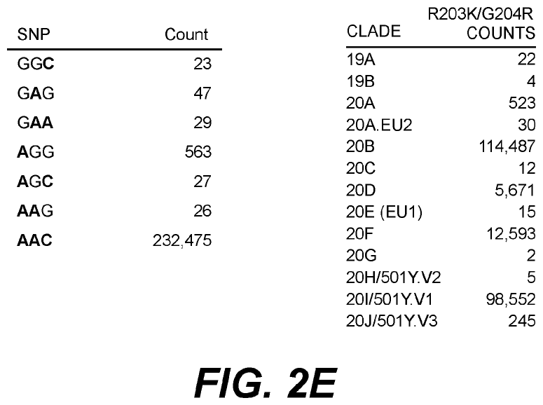

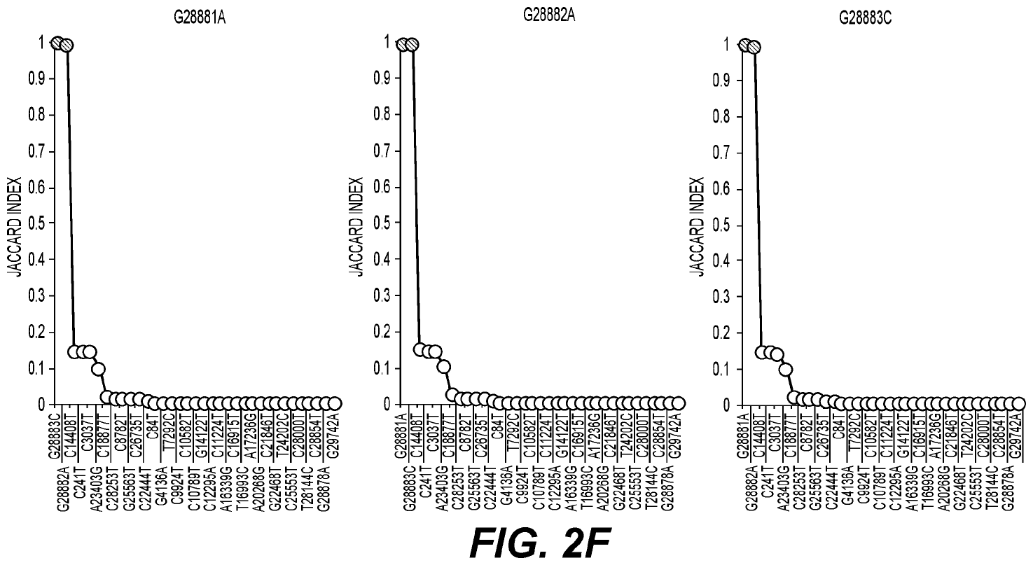

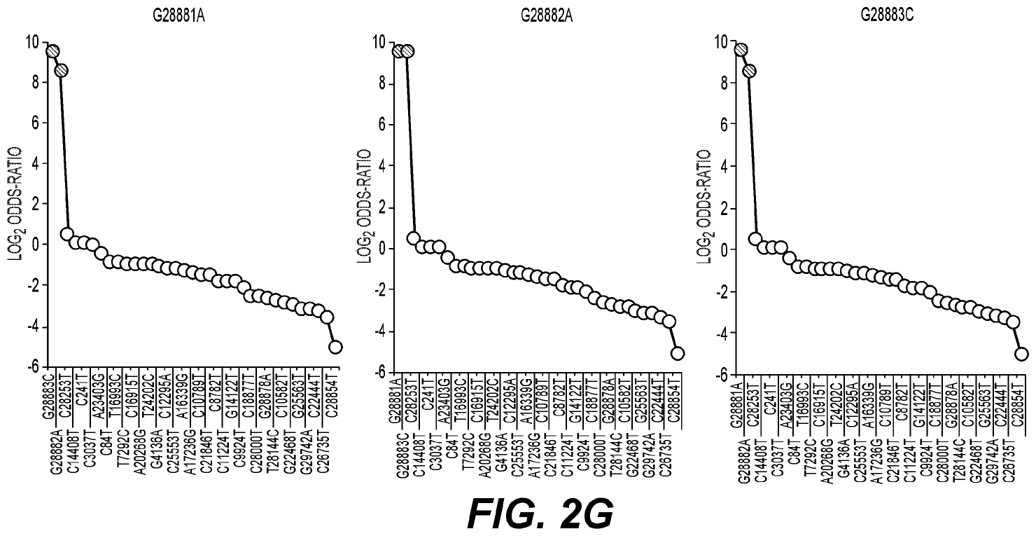

A total of 590K samples submitted to GISAID until February 24 were downloaded and SNPs identified by mapping against the Wuhan reference using minimap2⁷¹. The variants were queried to count the distribution of triplets among various Nextstrain clades (FIG. 2E). To identify if there are lineages of triplet SNPs in clades other than 20B, a phylogenetic tree was constructed by including all R203K/G204R samples found in other clades outside 20B and its subclades. As it was already evident that 20B and its subclades contains lineages of R203K/G204R samples, subsamples from 20B and its subclades were sufficient to obtain a total of 16,386 samples.

### Statistical Analysis

Statistical analyses were performed with the statistical software R version 4.0.3⁷² and the R package mgcv version 1.8.33.

### Plasmid and Cloning

The pLVX-EF1alpha-SARS-CoV-2-N-2xStrep-IRES-Puro was a gift from Nevan Krogan (Addgene plasmid #141391;
http://n2t.net/addgene:141391; RRID:Addgene_141391)³⁶. The three consecutive SNPs (G28881A, G28882A, G28883C), corresponding to N protein mutation sites R203K and G204R, were introduced by megaprime PCR mutagenesis using the primers listed in Table 1.

TABLE 1
Primers used for cloning

| Primer | |
|---|---|
| Name | Sequence 5′ to 3′ |
| pLVX-N1- | CTATTTCCGGTGAATTCGCCG |
| F1 | (SEQ ID NO: 2) |
| pLVX-N1- | GGGGGGGGATCCTTACTTTTC |
| R1 | (SEQ ID NO: 3) |
| pLVX-N1- | CCAGGGTCCAGTAAACGAACAAGTCCGGCGC |
| Mut-F1 | (SEQ ID NO: 4) |
| pLVX-N1- | GCGCCGGACTTGTTCGTTTACTGGACCCTGG |
| Mut-R1 | (SEQ ID NO: 5) |

2019-nCOV CDC Primers and Probe

| Name | Catalog# |
|---|---|
| nCOV_N1 Forward Primer | 10006821 |
| Aliquot, 50 nmol | |
| nCOV_N1 Reverse Primer | 10006822 |
| Aliquot, 50 nmol | |
| nCOV_N1 Probe Aliquot, | 10006823 |
| 25 nmol | |
| nCOV_N2 Forward Primer | 10006824 |
| Aliquot, 50 nmol | |
| nCOV_N2 Reverse Primer | 10006825 |
| Aliquot, 50 nmol | |
| nCOV_N2 Probe Aliquot, | 10006826 |
| 25 nmol | |
| E gene E_Sarbeco_F | ACAGGTACGTTAATAGTTAATAGCGT |
| | (SEQ ID NO: 6) |
| E_Sarbeco_R | ATATTGCAGCAGTACGCACACA |
| | (SEQ ID NO: 7) |
| E_Sarbeco_P1 | FAM-ACACTAGCCATCCTTACTGCGC |
| | TTCG-BBQ (SEQ ID NO: 8) |

### Cell Culture and Transfection

HEK293T (ATCC; CRL-3216) cells were grown in Dulbecco's modified Eagle's medium (DMEM) (4.5 g/l d-glucose and Glutamax, 1 mM sodium pyruvate) (GIBCO) and 1000 fetal bovine serum (FBS; GIBCO) with penicillin-streptomycin supplement, according to standard protocols (culture condition 37° C. and 500 CO2). Transfection often million cells per 15-cm dish with 2XStrep-tagged N plasmid (20 μg/transfection) was performed using lipofectamine-2000 according to standard protocol.

### Affinity Purification and On-Bead Digestion

Cell lysis and affinity purification with MagStrep beads (IBA Lifesciences) was manually performed according to the published protocol³⁶ with minor modifications. Briefly, after transfection (48 hours) cells were collected with 10 mM EDTA in 1×PBS and washed twice with cold PBS (lx). The cell pellets were stored at −80° C. Cells were lysed in lysis buffer (50 mM Tris-HCl, pH 7.4, 150 mM NaCl, 1 mM EDTA, 0.5% NP40, supplemented with protease and phosphatase inhibitor cocktails) for 30 minutes while rotating at 4° C. and then centrifuge at high speed to collect the supernatant. The cell lysate was incubated with prewashed MagStrep beads (30μl per reaction) for 3 hours at 4° C. The beads were then washed four times with wash buffer (50 mM Tris-HCl, pH 7.4, 150 mM NaCl, 1 mM EDTA, 0.05% NP40, supplemented with protease and phosphatase inhibitor cocktails) and then proceed with on-bead digestion. The on-bead digestion was carried out as described before³⁶. For affinity confirmation, bound proteins were eluted using buffer BXT (IBA Lifesciences) and after running on SDS-PAGE were subjected to silver staining and western-blot using anti-strep-II antibody (ab76949). To purify clean 2×Strep-tagged N protein (mutant and control), we applied stringent washing and double elution strategy.

### MS Analysis Using Orbitrap Fusion Lumos

The MS analysis was performed as described previously^{73,74} with slight modifications. For mass spectrometry analysis an Orbitrap Fusion mass spectrometer (MS) (Lumos, Thermo Fisher Scientific) was used in data-dependent acquisition (DDA) mode. For injection, 0.5 μg peptide mixture was used, and desalting was performed for 5 minutes in 0.1% FA in water. The gradient and all other steps were essentially the same as described⁷³.

Protein identification analysis from the raw mass spectrometry data was performed using the Maxquant software (version 1.5.3.30)⁷⁵ as described⁷³. For phosphorylated peptides, we used Maxquant label-free quantification (LFQ)⁷⁵. The analysis and quantification of phosphorylated peptides was performed according to published protocol⁷⁶.

### Analysis of Differential Interaction

The normalized LFQ data were processed for statistical analysis on the LFQ-Analyst a web-based tool⁷⁷ to performed pair-wise comparison between mutant and control N protein AP-MS data. The significant differentially changed proteins between mutant and control conditions were identified. The threshold cut-off of adjusted p-value<=0.05, and Log fold change>=1 were used. Among the replicates, outliers were removed based on correlation and PCA analysis. The GO enrichment analysis was performed on the LFQ-Analyst″.

### BS3 Cross-Linking

Bis(sulfosuccinimidyl) suberate (BS3, Thermo Scientific Pierce) was used for cross-linking of control and mutant N protein to analyze the oligomerization properties. The experiment was performed as reported previously⁷⁸.

### RNA-Sequencing and Differential Gene Expression Analysis

HEK293T cells were transfected with plasmids expressing the full-length N-control and N-mutant protein along with mock control. After 48-hour cells were harvested in Trizol and total RNA was isolated using Zymo-RNA Direct-Zol kit (Zymo, USA) according to the manufacture's instruction. The concentration of RNA was measured by Qubit (Invitrogen), and RNA integrity was determined by Bioanalyzer 2100 system (Agilent Technologies, CA, USA). The RNA was then subjected to library preparation using Ribozero-plus kit (Illumina). The libraries were sequenced on NovaSeq 6000 platform (Illumina, USA) with 150 bp paired-end reads.

The raw reads from HEK293T RNA-sequencing were processed and trimmed using trimmomatic⁶⁰ and mapped to annotated ENSEMBL transcripts from the human genome (hg19)^{79,80} using kallisto⁸¹. Differential expression analysis was performed after normalization using EdgeR integrated in the NetworkAnalyst⁸². GO biological process and pathway enrichment analyses on up-regulated genes were performed using NetworkAnalyst⁸²

### Daily Cases in Saudi Arabia

The numbers of Covid-19 cases registered in Saudi Arabian Cities were collected from The Ministry of Health Command Centre for COVID-19 (https://covid19.moh.gov.sa), Saudi Center of Disease Control and Prevention (https://covid19.cdc.gov.sa/), the Saudi Press Agency (SPA) (https://www.spa.gov.sa/search.php?lang=en&search=COVID), The Saudi Ministry of Interior (https://www.moi.gov.sa/), and Algaissi et al.¹.

### Polymorphisms

Duplicate reads were removed from the mapped short Illumina sequence reads using picard tools' MarkDuplicates function (Broad Institute, GitHub repository. http://broadinstitute.github.io/picard/). Apparent mismatches were collected from the output of samtools mpileup². Distal read positions were excluded and only read mapping and base calling qualities of at least 30 were considered. Positions with at a minimum coverage of 1000 and with an overall mismatch rate between 0.3 and 0.7 were considered as potential within-host polymorphic sites.

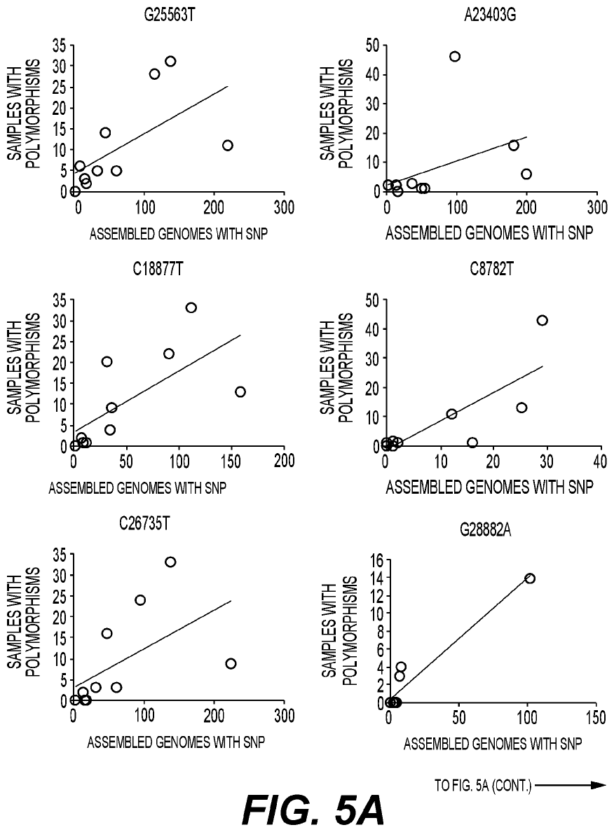

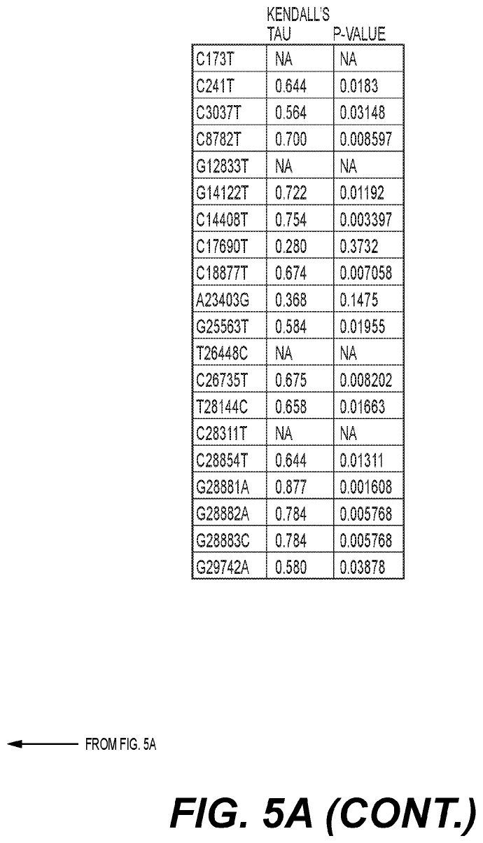

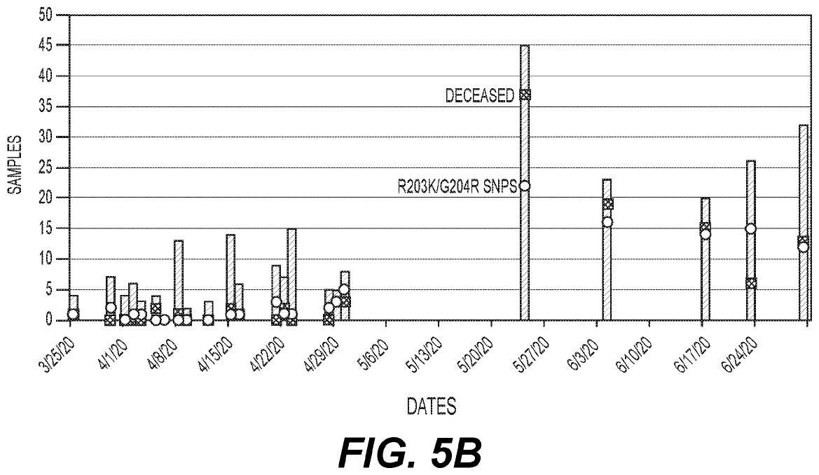

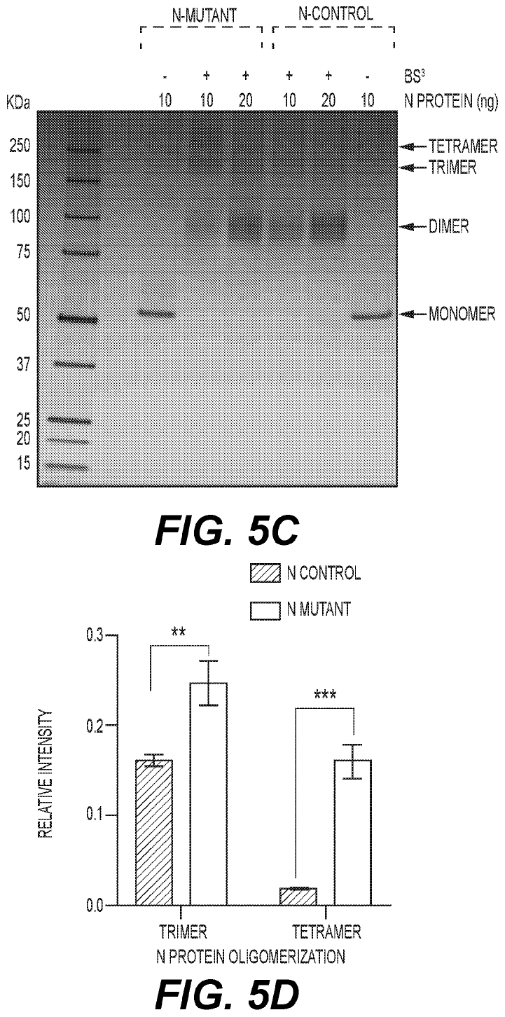

For each hospital, the number of samples with detected polymorphism at a given SNP site was plotted against the number of samples from the hospital that had this SNP in the assembled genome and the correlation was calculated. Examples of such plots for six different SNPs are shown in FIG. 5A.

We considered the frequency of a given SNP in the assembled virus genomes from a hospital as a proxy for the prevalence of this virus variant at the hospital. We further consider the probability of seeing polymorphisms of a given SNP due to co-infection as a product of the frequency of viruses with that SNP circulating among patients (the more prevalent a virus strain is in a given hospital environment the more likely it is to co-infect other COVID19 patients). Therefore, if observed polymorphisms were the result of co-infections, we would expect to see a positive correlation between SNP polymorphisms and SNP frequencies at different hospitals. In contrast, if observed polymorphisms were the result of inter-sample contaminations, such a correlation is not expected, as contamination would solely rely on the order and workflow by which the samples were handled.

The observed positive correlation between SNP polymorphisms and SNPs in assembled genomes between hospitals (FIG. 5A) is therefore consistent with the observed polymorphisms being the result of co-infection of patients. And we similarly consider this to be the case for the R203K/G204R SNPs.

### Ct Values

Although not associated with severity of infection, significantly lower Ct values have been reported for the D614G SNP³⁻⁵. In our experimental setup, samples were not only selected for further processing based on the initially obtained Ct values, but sample qPCRs were also run in separate batches, which produced separate standard curves. Additionally, two different laboratory kits were used for the qPCRs (see Methods). Furthermore, viral loads will also be a function of sampling time, with fluctuations over the course of infection and potential recovery. With these caveats in mind, we nevertheless tested for differences in Ct values between samples. This was done using samples processed with the TaqPath kit (see Methods). Hence, to prove that that the R203K/G204R SNPs result in higher viral loads, experimental in vitro infection studies with different virus genotypes are needed.

### Results and Discussion

SNP Calling and Phylodynamics of SARS-CoV-2 Samples from Saudi Arabia

SARS-CoV-2 genomes from 892 patient samples were sequenced and assembled. This group includes 144 patients that were placed in quarantine and had either mild symptoms or were asymptomatic. The remaining patients were all hospitalized. Data on comorbidities were available for 689 patients with diabetes (39%) and hypertension (35%) being the most abundant. Patient outcome data was available for 850 samples, and 199 patients (23%) died during hospitalization.

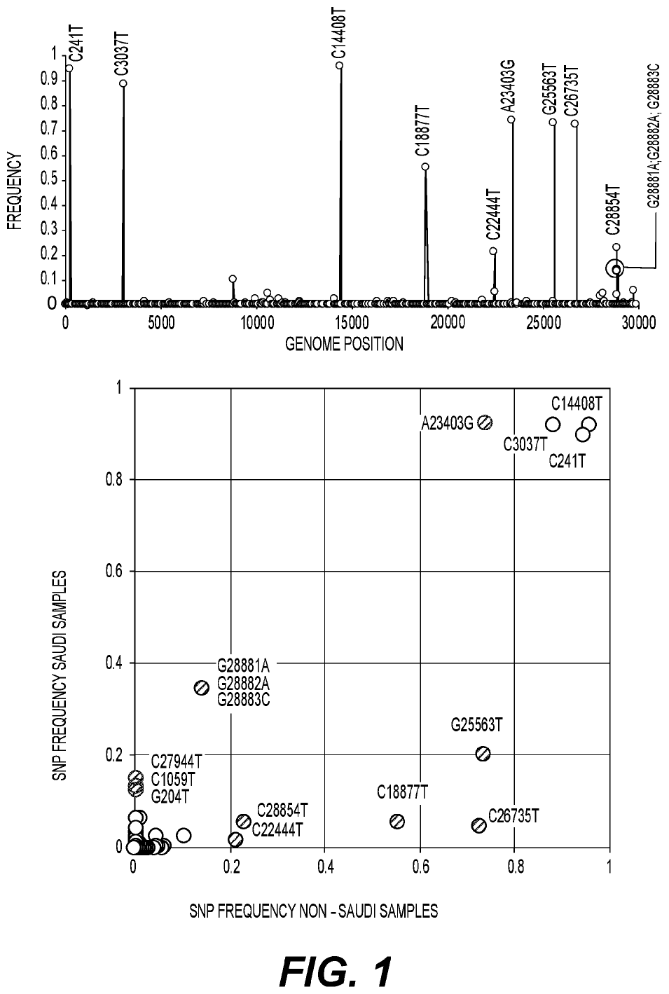

From the 892 assembled viral genomes collected over a period of 6 months, we found a total of 836 single-nucleotide polymorphisms (SNPs) compared to the Wuhan SARS-CoV-2 reference (GenBank accession: NC_045512) (FIG. 1). The observed numbers of SNPs relative to the Wuhan reference follow the numbers observed in global samples. Current studies further detected 41 indels (an insertion or deletion of bases in the genome) of which 26 reside in coding regions (Table 2).

TABLE 2
Detected Indels

| | | | | | sample |
|---|---|---|---|---|---|
| position | type | length | ref | allele | count |
| 103 | del | 2 | CTG | C | 2 |
| 509 | del | 9 | GGTCATGTTA (SEQ ID | G | 1 |
| | | | NO: 9 | | |
| 668 | del | 3 | AGTT | A | 3 |
| 685 | del | 9 | AAAGTCATTT (SEQ ID | A | 4 |
| | | | NO: 10) | | |
| 898 | ins | 262 | TTCATGCACTTTGTCCGAA | TTCATGCACTTTGTC | 1 |
| | | | CAACTGGACTTTATTGACA | CGAACAACTGGACTT | |
| | | | CTAAGAGGGGTGTATACTG | TATTGACACTAAGAG | |
| | | | CTGCCGTGAAC (SEQ ID | GGGTGTATACTGCTG | |
| | | | NO: 11) | CCGTGAACCACTTTT | |
| | | | | TCTTTGCATTTACTT | |
| | | | | TTTTATAGGAACTCC | |
| | | | | TGTCATCACTCTCTC | |
| | | | | ACACACACACTTAGA | |
| | | | | TGAACCTGATGGCTA | |
| | | | | CCCTCTTGAGTGCAT | |
| | | | | TAAAGACCTTCTAGC | |
| | | | | ACGTGCTGGTAAAGC | |
| | | | | CTCATGCACTTTGTC | |
| | | | | CGAACAACTGGACTT | |
| | | | | TATTGACACTAAGAG | |
| | | | | GGGTGTATACTGCTG | |
| | | | | CCGTGAAC (SEQ | |
| | | | | ID NO: 12) | |
| 2463 | del | 4 | TAAAA | TAA | 1 |
| 2628 | del | 3 | TTAT | TT | 1 |
| 2882 | ins | 282 | TGTGTTGTGGCAGATGCTG | TGTGTTGTGGCAGAT | 1 |
| | | | TC (SEQ ID NO: 13) | GCTGTCGTGTTGTGG | |
| | | | | CAGATGCTGTCGTGT | |
| | | | | TGTGGCAGATGCTGT | |
| | | | | CGTGTTGTGGCAGAT | |
| | | | | GCTGTCGTGTTGTGG | |
| | | | | CAGAGGCTGCTCGTG | |
| | | | | TTGTGGCAGATGCTG | |
| | | | | TCGTGTTGTGGCAGA | |
| | | | | TGCTGTCGTGTTGTG | |
| | | | | GCAGATGCTGTAGTG | |
| | | | | TTGCATCAGAGGCTG | |
| | | | | CTCGTGTTGTGGCAG | |
| | | | | ATGCTGTCGTGTTGT | |
| | | | | GGCAGATGCTGTCGT | |
| | | | | GTTGTGGCAGATGCT | |
| | | | | GTCGTGTTGTGGCAG | |
| | | | | ATGCTGTCGTGTTGT | |
| | | | | GGCAGATGCTGTC | |
| | | | | (SEQ ID NO: 14) | |
| 2882 | ins | 141 | TGTGTTGTGGCAGATGCTG | TGTGTTGTGGCAGAT | 1 |
| | | | TC (SEQ ID NO: 15) | GCTGTCGTGTTGTGG | |
| | | | | CAGATGCTGTCGTGT | |
| | | | | TGTGGCAGATGCTGT | |
| | | | | CGTGTTGTGGCAGAT | |
| | | | | GCTGTCGTGTTGTGG | |
| | | | | CAGATGCTGTCGTGT | |
| | | | | TGCATCAGAGGCTGC | |
| | | | | TCGTGTTGTGGCAGA | |
| | | | | TGCTGTC (SEQ ID | |
| | | | | NO: 16) | |
| 7626 | ins | 49 | ATTGTGATACATTCTGTGC | ATTGTGATACATTCT | 2 |
| | | | TGGTAGT (SEQ ID NO: | GTGCTGGTAGTTGTG | |
| | | | 17) | ATACATTCTGTGCTG | |
| | | | | GTAGT (SEQ ID | |
| | | | | NO: 18) | |
| 10535 | del | 27 | TACATGCACCATATGGAAT | T | 1 |
| | | | TACCAACTG (SEQ ID | | |
| | | | NO: 19) | | |
| 11074 | ins | 11 | CTTTTTTTT (SEQ ID | CTTTTTTTTTTT | 1 |
| | | | NO: 20) | (SEQ ID NO: 21) | |
| 18896 | ins | 309 | TTGTTAAGCGTGTTGACTG | TTGTTAAGCGTGTTG | 1 |
| | | | GACTATTGAATATCCTATA | ACTGGACTATTTAAT | |
| | | | ATTGGTGATGAACTGAAGA | ATCCTATAATTGGTG | |
| | | | TTAATGCGGCTTGTAGAAA | ATGAACTGAAGATTA | |
| | | | GGTTCAACA (SEQ ID | ATGCGGCTTGTAGAA | |
| | | | NO: 22) | AGGTTCAACATAACA | |
| | | | | TGTTGTGCCAACCAC | |
| | | | | CAGCACTCCTGGGAC | |
| | | | | CTCCACAGTGCACCT | |
| | | | | GGCAACCTCTGGGAC | |
| | | | | TCCATCCTCCCTGCC | |
| | | | | TGGCCACACAGCCCC | |
| | | | | TGTCCCTCTCTTGAT | |
| | | | | ACCATTCACCCTCAA | |
| | | | | CTTTACCAGATGGGA | |
| | | | | ATGTTAAGCGTGTTG | |
| | | | | ACTGGACTATTGAAT | |
| | | | | ATCCTATAATTGGTG | |
| | | | | ATGAACTGAAGATTA | |
| | | | | ATGCGGCTTGTAGAA | |
| | | | | AGGTTCAACA (SEQ | |
| | | | | ID NO: 23) | |
| 19517 | del | 3 | TGTA | T | 1 |
| 21066 | del | 5 | TAAAAA | TAA | 1 |
| 21561 | del | 2 | CAA | CA | 2 |
| 21624 | del | 33 | GAACTCAATTACCCCCTGC | G | 1 |
| | | | ATACACTAATTCTTT | | |
| | | | (SEQ ID NO: 24) | | |
| 21740 | del | 45 | TCCAATGTTACTTGGTTCC | TCCAATG | 1 |
| | | | ATGCTATACATGTCTCTGG | | |
| | | | GACCAATG (SEQ ID | | |
| | | | NO: 525) | | |
| 21781 | ins | 3 | CAA | CAAA | 1 |
| 21990 | del | 6 | TTTATTA | TTTA | 3 |
| 22048 | del | 3 | TGCG | T | 1 |
| 22288 | del | 6 | TGCTTTA | T | 1 |
| 22353 | del | 7 | CTTATTAT | CTTAT | 1 |
| 23701 | del | 1 | CA | C | 1 |
| 27263 | del | 29 | CTTTTAAAGTTTCCATTTG | CTT | 1 |
| | | | GAATCTTGATT (SEQ ID | | |
| | | | NO: 26) | | |
| 27694 | del | 8 | TTTCTTATT | TTT | 1 |
| 27697 | del | 5 | CTTATT | CTT | 4 |
| 28949 | del | 11 | AGATTGAACCAG (SEQ | A | 1 |
| | | | ID NO: 27) | | |
| 29727 | del | 22 | TTTCACCGAGGCCACGCGG | T | 1 |
| | | | AGTA (SEQ ID NO: | | |
| | | | 28) | | |
| 29755 | del | 1 | GA | G | 2 |
| 29774 | del | 18 | CTAGGGAGAGCTGCCTATA | CTA | 3 |
| | | | (SEQ ID NO: 29) | | |
| 29865 | ins | 5 | AA | AAAACA | 1 |
| 29865 | ins | 7 | AA | AAACAACA | 4 |
| 29865 | ins | 3 | AA | AACA | 1 |
| 29865 | ins | 9 | AA | AACCACAACA (SEQ | 1 |
| | | | | ID NO: 30) | |
| 29865 | ins | 10 | AA | AAGCCACAACA | 1 |
| 29866 | ins | 3 | A | AACC | 1 |
| 29866 | ins | 6 | A | AAGATGC | 1 |
| 29866 | ins | 9 | A | AAGCAGCCTC (SEQ | 1 |
| | | | | ID NO: 31) | |
| 29866 | ins | 3 | A | AATG | 1 |
| 29866 | ins | 8 | A | AGCAGATGC (SEQ | 1 |
| | | | | ID NO: 32) | |

Most indels were specific to a single sample, and no identical indel was found in more than four samples. Compared with global SNP data, seven SNPs were found in higher frequencies (absolute difference>0.1) in samples from Saudi Arabia (FIG. 1A). These include the Spike protein D614G (A23403G) and three consecutive SNPs causing the R203K and G204R changes in the nucleoprotein (G28881A, G28882A, and G28883C). Together with all sequences from Saudi Arabia available on GISAID on Dec. 31, 2020, the assembled sequences were used to construct the effective population size and growth rate estimates of SARS-CoV2 over the course of the first wave of the epidemic. The skygrowth model¹⁶ showed a downward trend in the effective reproduction number (R) over time with the timely introduction and maintenance of effective non-pharmaceutical interventions by the Saudi Ministry of Health. Following the lifting of restrictions towards the end of June, the model estimates that R remained below or at 1 to the end of the period covered by the genetic data presented in this study. The effective population size (Ne) represents the relative diversity of the sequences collected in Saudi Arabia over the course of the outbreak. The model predicts a peak in viral diversity at the beginning of June. This is ahead of the peak number of cases reported nationally and is likely influenced by the earlier peak in reported cases in the three cities, which contribute the most viral sequences to this analysis (Madinah, Makkah and Jeddah).

To simplify discussion of co-circulating virus variants, Nextstrain groups them into Clades, which are defined by specific combination of signature mutations. Clades are groups of related sequences that share a common ancestor. A maximum-likelihood phylogenetic analysis revealed that samples from Saudi Arabia represent 5 major Nextstrain clades¹², 19A-B and 20A-C. This highlighted the clade 20A that all carried the Nucleocapsid (N) protein R203K/G204R mutations¹⁷ with high incidences of ICU hospitalizations. These samples were predominantly coming from Jeddah.

Through time-scaled phylogenies dates of importation events were then estimated for each clade. The majority of importations for all clades were inferred to have occurred early in the outbreak, primarily in March and early April. Inferring importation events from a phylogenetic tree with estimated dating of nodes we see an early import from Asia followed by multiple imports from different continents.

Origin of R203K G204R SNPs and Importation into Saudi Arabia

A dated phylogeny of global samples showed that samples with the R203K/G204R SNPs are predominantly found in Nextstrain clades 20A, 20B and 20C, and do not form a monophyletic group. Furthermore, a few samples are further found in the early appearing 19A and 19B clades. However, due to the limited number of mutations separating SARS-CoV-2 genomes constructing a reliable and robust phylogeny is problematic¹⁸, and while different clades may be well supported, the exact relationship between clades is often less easily resolved. Although phylogenetic trees of SARS-CoV-2 genomes may appear to robustly reflect transmission events, collapsing branches with low support will typically result in extensive polytomies^{19,20} Additionally, the placement of individual virus genomes may be hampered by systematic errors, homoplasies, potential recombination, or co-infection of multiple virus strains^{18,19,21-23}. It is therefore not clear if the phylogenetic distribution of samples with R203K/G204R SNPs reflects multiple independent origins of the SNPs, although it is evident that the R203K/G204R SNPs appeared early in the pandemic spread. Consistent with this, we find the earliest estimated importation events of R203K/G204R SNPs in late January 2020, most likely from Italy. This thus suggest a slightly earlier importation date than the estimate of importation events of clade 20B. Within our sampling window we observe an apparent transient increase in the frequency of R203K/G204R SNPs (FIG. 2A) in accordance with earlier observations^{17,24}. This peak is similarly observed in global data up until the fall of 2020, where the R203K/G204R SNPs once again increase along with the Spike protein Y501N mutation in the B1.1.17 lineage²⁵ (FIG. 2A).

A Mutant Form of the Nucleocapsid (N) Protein Associated with Higher Viral Loads in COVID-19 Patients in Saudi Arabia

A genome-wide association study between SARS-CoV-2 SNPs and patient mortality identified the three consecutive SNPs (G28881A, G28882A, G28883C) underlying the R203K/G204R mutations (FIG. 2B, FIG. 2D). Of the 892 assembled genomes, 882 (98.9%) genomes either have the three reference alleles, GGG, or the three mutant alleles, AAC, at positions 28,881-28,883. This is similarly found in global samples deposited in GIASID in 2020, where 99.7% of samples with SNPs at positions 28,881-28,883 contain all three SNPs (FIG. 2E). In our samples, no other SNPs co-occur with the R203K/G204R SNPs (FIG. 2F-G). The frequency of the R203K/G204R SNPs is markedly higher in samples from Jeddah, where the observed frequency of 0.38 is more than 10-fold higher than the average of the other cities. Within-host polymorphism has been observed for the R203K/G204R SNPs either resulting from co-infection of multiple strains or cross-sample contamination²¹. Co-infection of SARS-CoV-2 is demonstrated through observations of recombination between genetically distinct lineages²⁶. To rule out cross-sample contamination, we investigated the levels of within-host polymorphisms in a range of SNP positions and found this more consistent with cases of co-infection among patients rather than contamination issues.

Using multivariable regression, subsequent studies evaluated the effect of the R203K/G204R SNPs on mortality, severity, and viral load in COVID-19 patients' samples for which limited amount of clinical meta-datasets were available. Disease severity was defined as deceased patients and patients admitted to ICU.

For mortality and severity, studies first fitted a linear model using R203K/G204R SNPs as a covariate. Then we fitted adjusted models by including gender, age, comorbidities, hospital, and time. 12 additional SNPs (C241T, C1191T, C3037T, G10427A, C14408T, C15352T, C18877T, A23403G, G25563T, C26735T, T27484C, and C28139T) that co-occurred with the R203K/G204R SNPs in at least five samples in the model were included. The A23403G mutation results in the Spike protein D614G SNP that is associated with higher viral load¹³ was included. Age and time were included using smoothing splines to allow for potential non-linear relationships²⁷

Using an unadjusted logistic regression model that did not include time, a positive and statistically significant association between R203K/G204R SNPs and severity was observed. Specifically, he log-odds of severity increased by 1.18, 95% CI 0.22-2.13. A positive significant association was also observed for the C14408T SNP, and a negative association for the C241T SNP

In the time-adjusted model, the log-odds for the R203K/G204R SNPs increased to 1.38, 95% CI 0.28-2.48. In this model, the C241T SNP again displayed a significant negative association, and a positive association was now observed for the C1887T SNP.

The relationship between mortality and R203K/G204R SNPs was positive and statistically significant in the model that did not include time with log-odds equal to 1.04, 95% 0.16-1.92. No significant association was observed for other SNPs. However, after adjusting for time as a variable, there was no longer any association between R203K/G204R SNPs and mortality (log-odds: 0.58, 95% CI-0.41-1.56). The models thus suggest a temporal component in the observations, and it is important to note that the recorded mortalities from Jeddah are concentrated on just a few dates (FIG. 5B).

Subsequent studies evaluated if R203K/G204R SNPs were associated with higher viral copy numbers as indicated by the cycle threshold (Ct) values obtained through quantitative PCRs. As two different kits were used for the qPCR reactions (see Methods), we fitted adjusted models that besides sex, age, comorbidities, hospital, and time, included qPCR kits and the above-mentioned SNPs as covariates. From this adjusted regression a positive and statistically significant relationship was observed between R203K/G204R SNPs and log10(viral copy number), with the mean of log10(viral copy number) values increasing by 1.33 units (95% CI 0.72-1.93). Similarly, the model showed a positive significant association between the SNPs A23403G (Spike protein D614G) and C26735T SNPs and log10(viral copy number), the former being consistent with earlier reports^{13,30}. A significant negative association was found for the C3037T C14408T, and G25563T SNPs. The positive and statistically significant association of R203K/G204R SNPs with higher viral load in critical COVID-19 patients indicating their functional implications during viral infection.

The R203K G204R Mutations in the N Protein Affect its Interaction with Host Proteins

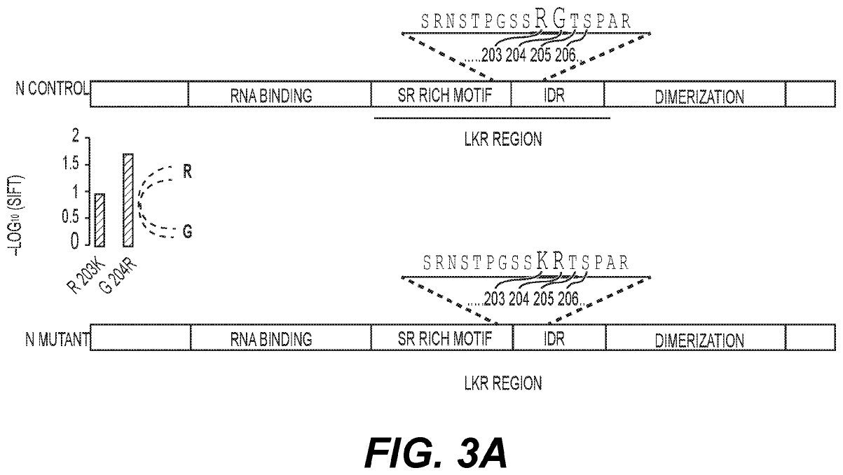

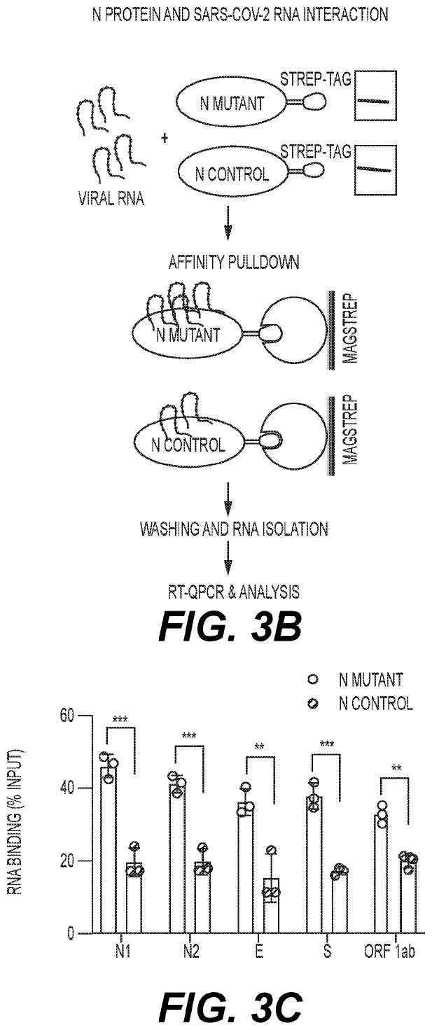

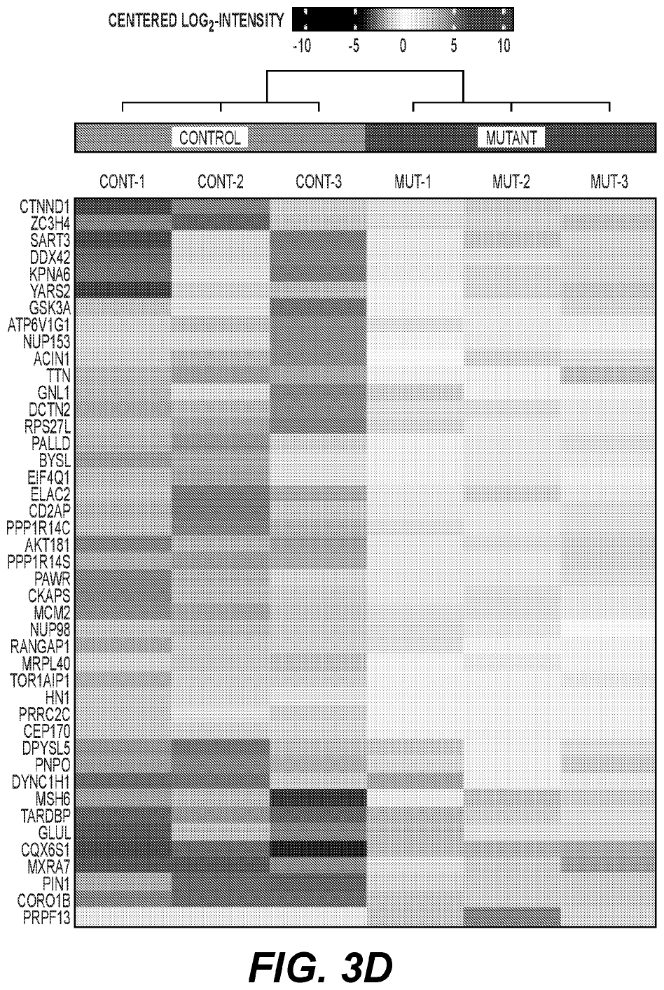

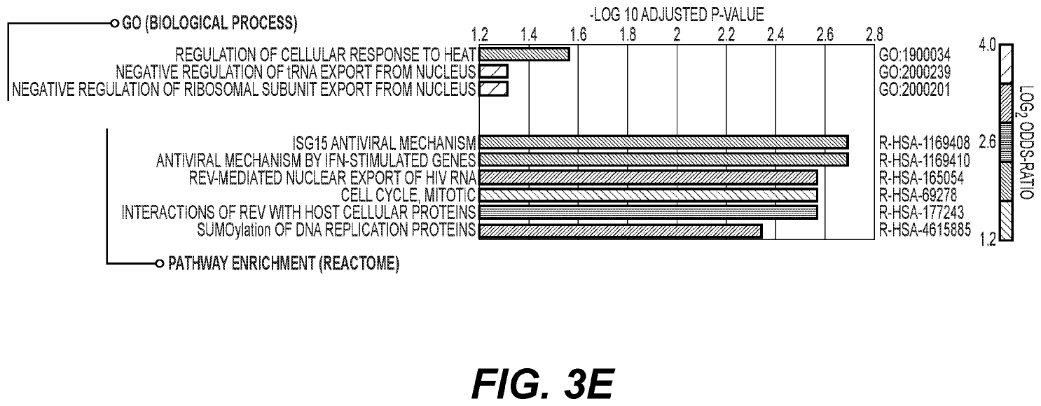

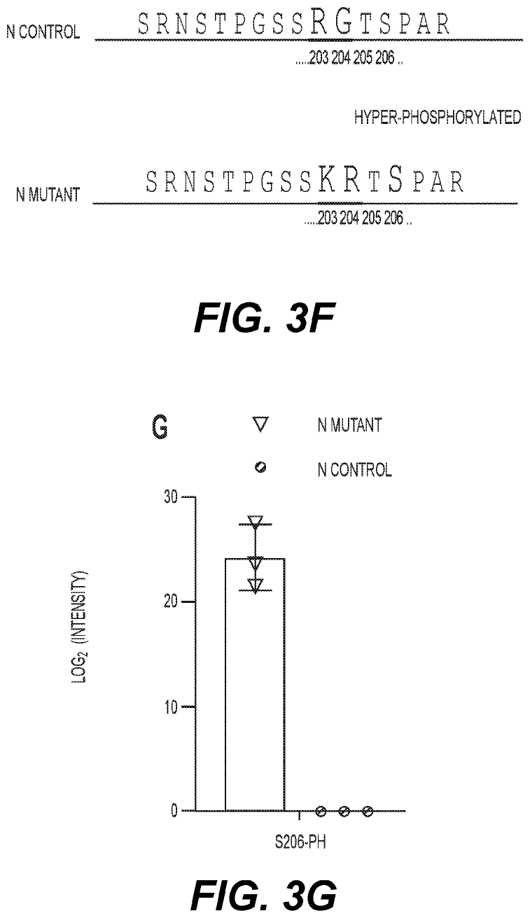

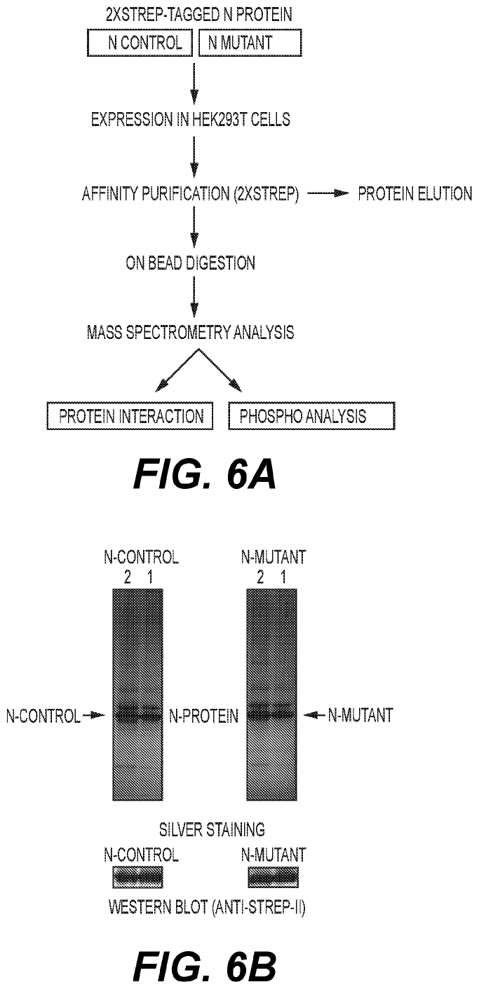

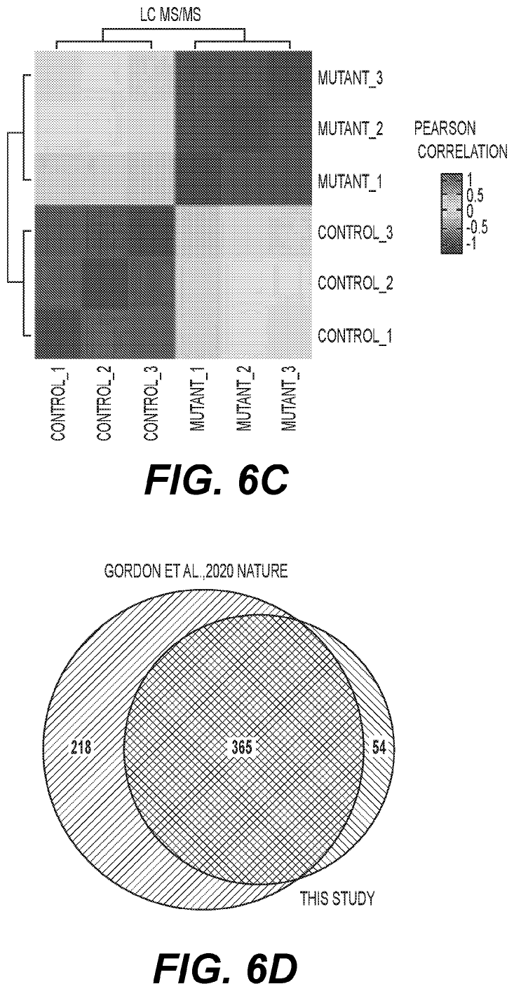

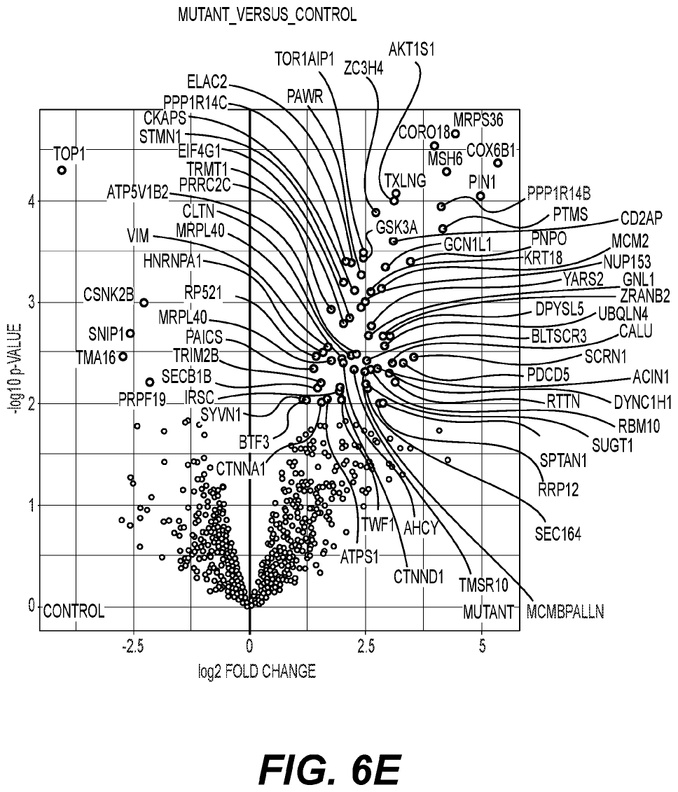

According to the SIFT tool²⁸, a substitution at position 204 from G to R in the N protein is predicted to affect functional properties (FIG. 3A). Therefore, subsequent studies investigated how the two amino acids substitution (R203K and G204R) in the N protein impact its functional interaction with the host that could modulate viral pathogenesis and rewiring of host cell pathways and processes. HEK-293T cells (3 biological replicates,) were used for affinity-purification followed by mass spectrometry analysis (AP-MS) to identify host proteins associated with the control and mutant N protein (FIG. 6A-D). The majority (87%) of non-differentially interacting proteins overlapped with the previously reported³⁵ N protein interacting partners (FIG. 6D).

43 human proteins that displayed significant (adjusted p-value<0.05, and Log2 fold change≥1) differential interactions with the mutant and control N protein were identified (FIG. 3D, FIG. 6E, Table 3).

TABLE 3
Proteins displaying significant differential interactions

| | | mutant_vs_con- | | | |
|---|---|---|---|---|---|
| Gene | Protein | trol_log | mutant_vs_con- | mutant_vs_con- | |
| Name | IDs | 2 fold change | trol_p.val | trol_significant | |
| ACIN1 | Q9UKV3 | 2.68 | 0.0014 | TRUE | Apoptotic chromatin condensation |
| | | | | | inducer in the nucleus |
| AKT1S1 | Q96B36 | 3.87 | 2.56E−05 | TRUE | Proline-rich AKT1 substrate 1 |
| CD2AP | Q9Y5K6 | 3.08 | 0.000245 | TRUE | CD2-associated protein |
| CKAP5 | Q14008 | 2.18 | 0.0013 | TRUE | Cytoskeleton-associated protein 5 |
| CORO1B | Q9BR76 | 4.76 | 0.000167 | TRUE | Coronin-1B |
| COX6B1 | P14854 | 5.5 | 3.51E−06 | TRUE | Cytochrome c oxidase subunit 6B1 |
| CSNK2B | P67870 | −2.82 | 0.000532 | TRUE | Casein kinase ∥ subunit beta |
| CTNND1 | O60716 | 2.3 | 0.000923 | TRUE | Catenin delta-1 |
| DPYSL5 | Q9BPU6 | 3.8 | 0.000207 | TRUE | Dihydropyrimidinase-related protein 5 |
| ELAC2 | Q9BQ52 | 2.61 | 0.000164 | TRUE | Zinc phosphodiesterase ELAC protein 2 |
| GCN1L1 | Q92616 | 2.76 | 0.00025 | TRUE | Translational activator GCN1 |
| HN1 | Q9UK76 | 1.76 | 0.00123 | TRUE | Hematological and neurological |
| | | | | | expressed 1 protein |
| KRT18 | P05783 | 2.63 | 0.000766 | TRUE | Keratin, type I cytoskeletal 18 |
| MCMBP | Q9BTE3 | 1.88 | 0.00116 | TRUE | Mini-chromosome maintenance complex- |
| | | | | | binding protein |
| MRPL40 | Q9NQ50 | 2.92 | 0.0016 | TRUE | 39S ribosomal protein L40, mitochondrial |
| MRPS36 | P82909 | 4.31 | 0.00034 | TRUE | 28S ribosomal protein S36, mitochondrial |
| MSH6 | P52701 | 3.63 | 0.000413 | TRUE | DNA mismatch repair protein Msh6 |
| NUP153 | P49790 | 2.27 | 0.000366 | TRUE | Nuclear pore complex protein Nup153 |
| PALLD | Q8WX93 | 2.94 | 0.000289 | TRUE | Palladin |
| PAWR | Q96IZ0 | 2.58 | 0.00031 | TRUE | PRKC apoptosis WT1 regulator protein |
| PIN1 | Q13526 | 3.71 | 0.00102 | TRUE | Peptidyl-prolyl cis-trans isomerase |
| | | | | | NIMA-interacting 1 |
| PNPO | Q9NVS9 | 3.13 | 0.00111 | TRUE | Pyridoxine-5-phosphate oxidase |
| PPP1R14B | Q96C90 | 3.61 | 0.000247 | TRUE | Protein phosphatase 1 regulatory subunit 14B |
| PPP1R14C | Q8TAE6 | 2.74 | 0.000809 | TRUE | Protein phosphatase 1 regulatory subunit 14C |
| PRPF19 | Q9UMS4 | −1.89 | 0.000803 | TRUE | Pre-mRNA-processing factor 19 |
| PRRC2C | E7EPN9 | 1.76 | 0.00119 | TRUE | Protein PRRC2C |
| PTMS | P20962 | 4.09 | 0.000322 | TRUE | Parathymosin |
| RANGAP1 | P46060 | 2.39 | 0.00113 | TRUE | Ran GTPase-activating protein 1 |
| RBM10 | P98175 | 2.41 | 0.00211 | TRUE | RNA-binding protein 10 |
| STMN1 | P16949 | 2.03 | 0.000652 | TRUE | Stathmin |
| TMA16 | Q96EY4 | −3.23 | 0.000435 | TRUE | Translation machinery-associated protein 16 |
| TOP1 | P11387 | −3.46 | 0.000197 | TRUE | DNA topoisomerase 1 |
| TOR1AIP1 | Q5JTV8-3 | 2.46 | 0.000327 | TRUE | Torsin-1A-interacting protein 1 |
| YARS2 | Q9Y2Z4 | 3.17 | 0.000165 | TRUE | Tyrosine--tRNA ligase, mitochondrial; |
| | | | | | Tyrosine--tRNA ligase |
| ZC3H4 | Q9UPT8 | 3.64 | 0.000483 | TRUE | Zinc finger CCCH domain-containing |
| | | | | | protein 4 |
| NUP98 | P52948 | 1.49 | 0.00712 | TRUE | Nuclear pore complex protein Nup98- |
| | | | | | Nup96; Nuclear pore complex protein |
| | | | | | Nup98; Nuclear pore complex protein Nup96 |
| ATP6V1B2 | P21281 | 1.87 | 0.00453 | TRUE | V-type proton ATPase subunit B, |
| | | | | | brain isoform |
| ZRANB2 | O95218 | 2.97 | 0.00258 | TRUE | Zinc finger Ran-binding domain-containing |
| | | | | | protein 2 |
| VIM | P08670 | 1.7 | 0.00279 | TRUE | Vimentin |
| TXLNG | Q9NUQ3 | 2.69 | 0.00396 | TRUE | Gamma-taxilin |
| SYVN1 | Q86TM6 | 1.46 | 0.00976 | TRUE | E3 ubiquitin-protein ligase synoviolin |
| SNIP1 | Q8TAD8 | −2.66 | 0.00396 | TRUE | Smad nuclear-interacting protein 1 |
| OGFR | Q9NZT2 | 1.55 | 0.00719 | TRUE | Opioid growth factor receptor |
| MCM2 | P49736 | 1.52 | 0.00511 | TRUE | DNA replication licensing factor MCM2 |
| DDX23 | Q9BUQ8 | 1.49 | 0.00124 | TRUE | Probable ATP-dependent RNA helicase |
| | | | | | DDX23 |
| EPB41 | P11171 | 1.62 | 0.00538 | TRUE | Protein 4.1 |
| HNRNPA1 | P09651 | 1.59 | 0.00319 | TRUE | Heterogeneous nuclear ribonucleoprotein |
| | | | | | A1; Heterogeneous nuclear ribonucleoprotein |
| | | | | | A1, N-terminally processed |
| GLUL | P15104 | 2.8 | 0.00715 | TRUE | Glutamine synthetase |
| GNL1 | P36915 | 2.33 | 0.00459 | TRUE | Guanine nucleotide-binding protein-like 1 |

Among these, 42 proteins showed increased interaction and one protein (PRPF19) showed decreased interaction with the N mutant (FIG. 3D, FIG. 6E). Among the group with increased interaction, many are proteins associated with TOR and other signaling pathways (such as AKT1S1 and PIN1), proteins associated with the viral process, viral transcription, and negative regulation of RNA nuclear export (NUP153 and NUP98), and proteins involved in apoptotic and cell death processes (PAWR and ACIN1). Proteins were also identified in the mutant condition that are linked with the immune system processes (PTMS), kinase activity (GCN1), and translation (e.g., MRPS36). In the group with decreased interaction, SNIP1 (NF-kappaB signaling), TMA16 (translation), and CSNK2B (casein kinase II) were identified (FIG. 3D, FIG. 6E). Gene ontology analysis showed that the most enriched biological processes are associated with negative regulation of tRNA and ribosomal subunit export from the nucleus (FIG. 3E).

### N Mutant Protein has High Oligomerization Potential and RNA Binding Affinity

The SARS-CoV-2 N protein binds the viral RNA genome and is central to viral replication³⁰. Protein structure predictions have shown that the R203K/G204R mutations result in significant changes in protein structure²⁴, theoretically destabilizing the N structure³¹, and potentially enhancing the protein's ability to bind RNA and alter its response to serine phosphorylation events³². The R203K/G204R mutations in the SARS-CoV-2 N protein are within the linkage region (LKR) containing the serine/arginine-rich motif (SR-rich motif) (FIG. 3A), known to be involved in the oligomerization of N proteins^{33,34}. Protein cross-linking shows that N mutant protein (with the R203K/G204R mutations) has higher oligomerization potential compared to the control N protein (without the changed amino acids) at low protein concentration (FIGS. 5C-D).

Given that the oligomerization of N protein acts as a platform for viral RNA interactions³⁵, we sought to examine the binding affinity of mutant and control N protein with viral RNA isolated from COVID-19 patient swabs. The RNA-binding activity of mutant and control N proteins was examined by pulled-down viral RNA through an in vitro RIP assay (FIG. 3B), and our data revealed that the mutant N protein enriched significantly higher level of viral RNA compared to control protein (FIG. 3C). This indicates a strong binding capability of mutant N proteins with viral RNA, which could potentially impact the essential roles of N protein at various stages of viral life cycle and its interaction with the host.

The R203K G204R Mutations in the Nprotein Affect its Interaction with Host Proteins

According to the SIFT tool²⁹, a substitution at position 204 from G to R in the N protein is predicted to affect functional properties (FIG. 3A). Therefore, we decided to investigate how the two amino acids substitution (R203K and G204R) in the N protein impact its functional interaction with the host that could modulate viral pathogenesis and rewiring of host cell pathways and processes. HEK-293T cells (3 biological replicates) were used for affinity-purification followed by mass spectrometry analysis (AP-MS) to identify host proteins associated with the control and mutant N protein (FIG. 6A-D). The majority (87%) of non-differentially interacting proteins overlapped with the previously reported³⁶ N protein interacting partners. We identified 48 human proteins that displayed significant (adjusted p-value≤0.05, and Log2 fold change≥1) differential interactions with the mutant and control N protein (FIG. 3D, FIG. 6E, Table 3). Among these, 43 proteins showed increased interaction and 5 proteins showed decreased interaction with the N mutant. Among the group with increased interaction, we identified many proteins associated with TOR and other signaling pathways (such as AKT1S1 and PIN1), proteins associated with the viral process, viral transcription, and negative regulation of RNA nuclear export (NUP153 and NUP98), and proteins involved in apoptotic and cell death processes (PAWR, and ACIN1) (FIG. 3D, FIG. 6E). We also identified proteins in the mutant condition that are linked with the immune system processes (PTMS), kinase activity (GCN1), and translation (e.g., MRPS36) (FIG. 3D, FIG. 6E). In the group with decreased interaction, we identified SNIP1 (NF-kappaB signaling), TMA16 (translation), and CSNK2B (casein kinase II6)6. Gene ontology analysis showed that the most enriched biological processes are associated with negative regulation of tRNA and ribosomal subunit export from the nucleus (FIG. 3E). This finding suggests that the mutant virus may more efficiently inhibit and hijack the host translation to facilitate viral replication and pathogenesis. Further, many viruses can manipulate the host sumoylation process to enhance viral survival and pathogenesis³⁷. By pathway enrichment analysis of differentially interacting proteins, we identified pathways associated with the sumoylation of host proteins and antiviral mechanisms (FIG. 3E).

### Serine 206 (S206) Displays Hyper-Phosphorylation in the Mutant N Protein

To further understand the functional relevance of KR mutation in the N protein, performed phosphoproteomic analysis were performed in control and mutant conditions. The data consistently showed that the serine 206 (S206) site, which is next to the KR mutation site (FIG. 3F), is highly phosphorylated, specifically in the mutant N protein (FIG. 3G, Table 4).

TABLE 4
Phosphorylation

| | | | Intensity of phosphorylation | | | | | |
|---|---|---|---|---|---|---|---|---|
| Protein | | Localization | N mutant | N mutant | N mutant | N control | N control | N control |
| Name | phosphosite | prob | (Rep1) | (Rep2) | (Rep3) | (Rep1) | (Rep2) | (Rep3) |
| Nucleo- | S206 | 0.994527 | 207590000 | 13224000 | 2966400 | 0 | 0 | 0 |
| capsid (N) | | | | | | | | |
| Nucleo- | S2 | 1 | 955740 | 1907100 | 2265000 | 1921900 | 2763700 | 3502800 |
| capsid (N) | | | | | | | | |
| Nucleo- | S79 | 0.969512 | 1327000 | 1562200 | 2342900 | 2651100 | 2688800 | 2985200 |
| capsid (N) | | | | | | | | |
| Nucleo- | S180 | 0.997001 | 28745000 | 41617000 | 50307000 | 35401000 | 43784000 | 47220000 |
| capsid (N) | | | | | | | | |
| Nucleo- | S176 | 0.9764 | 15311000 | 11139000 | 12863000 | 13966000 | 18277000 | 18382000 |
| capsid (N) | | | | | | | | |

Log2 of Intensities (related to FIG. 3G)

| | N-mutant | | | N-control | | |
|---|---|---|---|---|---|---|
| S206-ph | 27.6291617 | 23.6566553 | 21.5002817 | NA | NA | NA |
| S2-ph | 19.8662587 | 20.8629491 | 21.1110796 | 20.8741018 | 21.3981696 | 21.7400772 |
| S79-ph | 20.3397369 | 20.5751477 | 21.1598639 | 21.3381597 | 21.358531 | 21.5093962 |
| S180-ph | 24.7768077 | 25.3106696 | 25.5842558 | 25.0772868 | 25.3839004 | 25.4928947 |
| S176-ph | 23.8680652 | 23.4091164 | 23.6167238 | 23.7354155 | 24.1235259 | 24.1317904 |

Notably, the phosphorylation level at serine 2 (S2) and other serine sites (S79, S176, and S180) within the LKR region did not change between mutant and control conditions (FIG. 3F).

### The N Mutant (R203K G204R) Induces Overexpression of Immune Related Genes in Transfected Host Cells

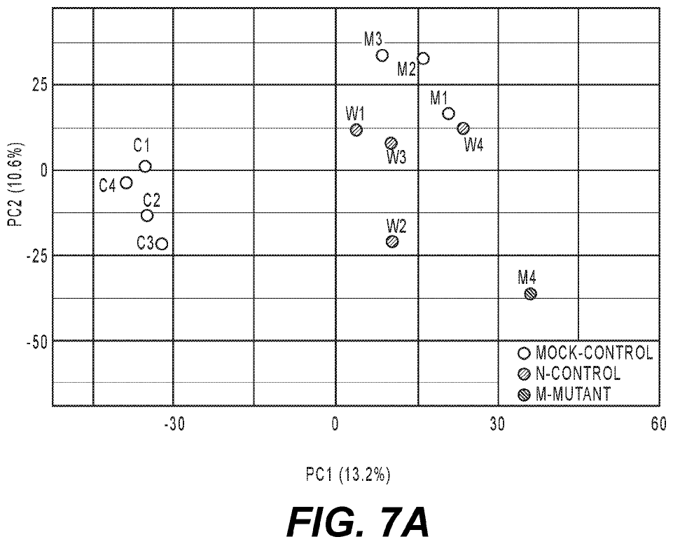

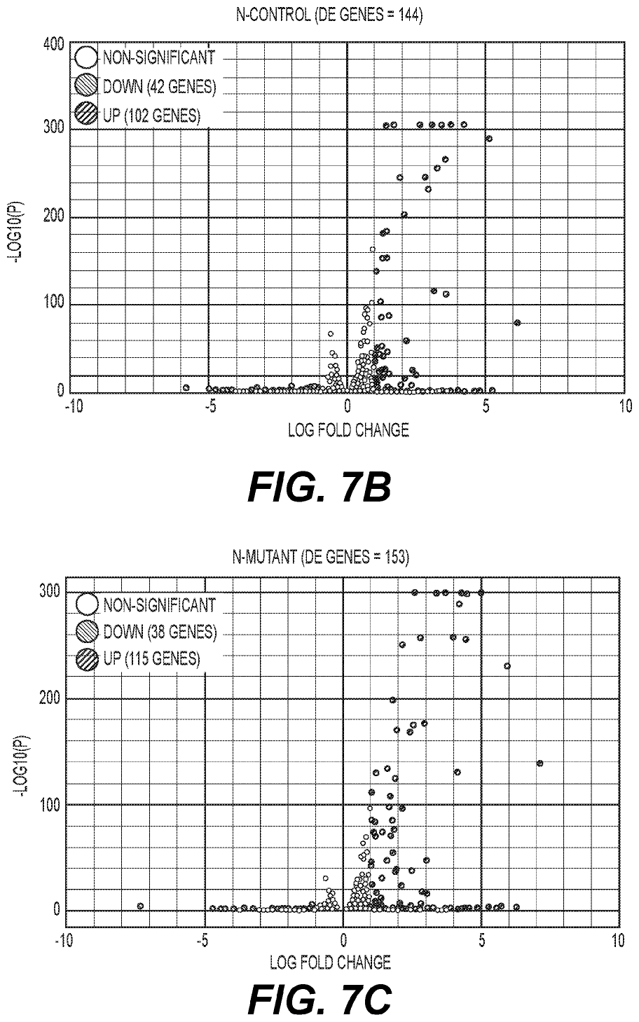

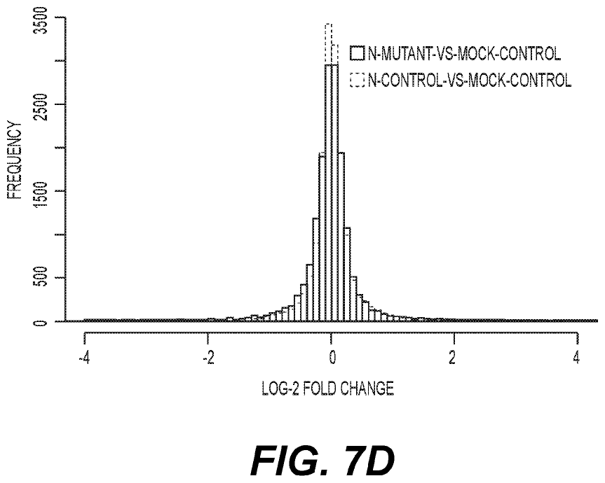

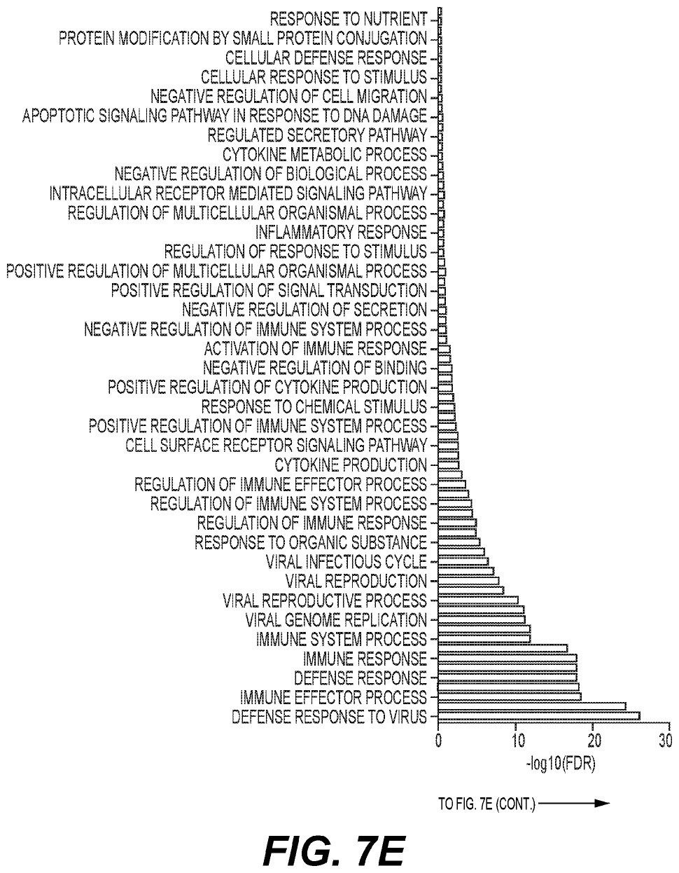

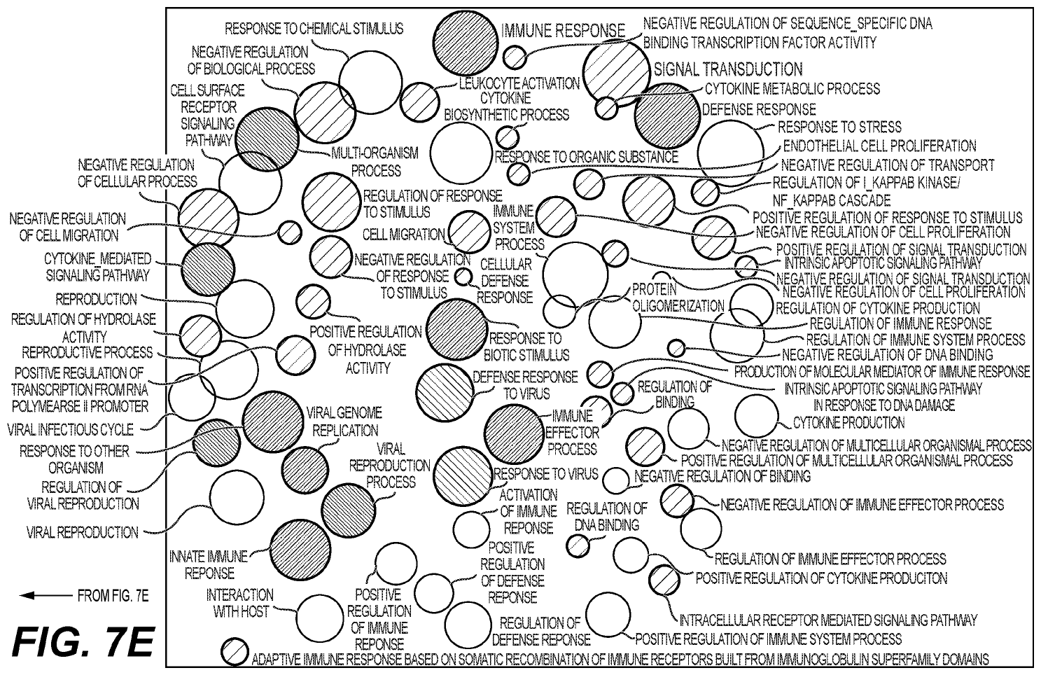

To understand whether the R203K/G204R mutations in the N gene affect host cell transcriptome, HEK293T cells were transfected with plasmids expressing the full-length N-control and N-mutant protein along with mock-transfection control. The transcriptome profile of N-mutant and N-control transfected cells displays a distinct pattern from the mock-control (FIG. 7A). 144 and 153 differentially expressed (DE) genes were identified in the N-control and N-mutant transfected cells, respectively, with adjusted p-value≤0.05 and log2 fold-change≥1 (FIGS. 7B-C and Table 5).

TABLE 5
Differentially expressed genes

| logFC | logFC | | | | adj. | | |
|---|---|---|---|---|---|---|---|
| (N-mutant) | (N-control) | logCPM | LR | PValue | P.Val | Symbols | Name |
| 6.5909 | 5.7375 | 5.548 | 569.36 | 2.32E−124 | 9.22E−121 | IFI44 | interferon induced protein 44 |
| 4.7401 | 3.9182 | 6.2177 | 499.18 | 4.01E−109 | 9.13E−106 | IFITM1 | interferon induced transmembrane protein 1 |
| 4.5185 | 3.8033 | 6.1309 | 710.47 | 5.28E−155 | 2.81E−151 | IFI44L | interferon induced protein 44 like |
| 4.4642 | 3.5178 | 6.8124 | 439.92 | 2.96E−96 | 5.25E−93 | IFIT3 | interferon induced protein with |
| | | | | | | | tetratricopeptide repeats 3 |
| 4.2358 | 3.4523 | 7.2185 | 847.95 | 7.42E−185 | 1.18E−180 | IFI6 | interferon alpha inducible protein 6 |
| 4.0804 | 3.3747 | 6.3073 | 756.23 | 6.11E−165 | 4.87E−161 | BST2 | bone marrow stromal cell antigen 2 |
| 3.9799 | 3.1183 | 4.7279 | 228.02 | 3.07E−50 | 2.33E−47 | RSAD2 | radical S-adenosyl methionine domain containing 2 |
| 3.9792 | 2.9881 | 3.5638 | 123.98 | 1.20E−27 | 6.81E−25 | OAS1 | 2′-5′-oligoadenylate synthetase 1 |
| 3.9121 | 3.1594 | 6.5215 | 347.03 | 4.41E−76 | 5.02E−73 | IFIT2 | interferon induced protein with |
| | | | | | | | tetratricopeptide repeats 2 |
| 3.8331 | 3.0151 | 8.0227 | 516.7 | 6.31E−113 | 1.68E−109 | IFIT1 | interferon induced protein with |
| | | | | | | | tetratricopeptide repeats 1 |
| 3.7219 | 2.9716 | 6.2972 | 431.3 | 2.21E−94 | 3.52E−91 | ISG15 | ISG15 ubiquitin like modifier |
| 3.1787 | 2.7725 | 1.3727 | 20.032 | 4.47E−05 | 0.0036888 | CXCL10 | C—X—C motif chemokine ligand 10 |
| 3.0411 | 2.3176 | 7.3672 | 408.78 | 1.71E−89 | 2.27E−86 | OAS3 | 2′-5′-oligoadenylate synthetase 3 |
| 2.7304 | 1.526 | 4.3908 | 36.542 | 1.16E−08 | 2.37E−06 | PARP10 | poly(ADP-ribose) polymerase family member 10 |
| 2.6618 | 2.2041 | 6.6264 | 549.98 | 3.74E−120 | 1.19E−116 | DDX60 | DExD/H-box helicase 60 |
| 2.5985 | −1.0584 | 0.74694 | 22.054 | 1.63E−05 | 0.0015893 | RPL37P6 | ribosomal protein L37 pseudogene 6 |
| 2.5804 | 1.7983 | 4.6689 | 221.45 | 8.19E−49 | 5.93E−46 | CMPK2 | cytidine/uridine monophosphate kinase 2 |
| 2.5427 | 1.9862 | 8.1562 | 484.13 | 7.45E−106 | 1.48E−102 | DDX58 | DExD/H-box helicase 58 |
| 2.5298 | 2.5444 | 2.3412 | 24.707 | 4.31E−06 | 0.00051599 | HSD17B13 | hydroxysteroid 17-beta dehydrogenase 13 |
| 2.4544 | 1.7193 | 6.6465 | 243.69 | 1.21E−53 | 1.01E−50 | IFITM3 | interferon induced transmembrane protein 3 |
| 2.3399 | 1.917 | 1.698 | 17.42 | 0.00016493 | 0.011042 | SP140 | SP140 nuclear body protein |
| 2.3349 | 1.7178 | 6.5483 | 235 | 9.34E−52 | 7.44E−49 | HELZ2 | helicase with zinc finger 2 |
| 2.2868 | 1.9946 | 4.0167 | 82.703 | 1.10E−18 | 4.74E−16 | SAMD9L | sterile alpha motif domain containing 9 like |
| 2.2536 | 1.4984 | 3.3321 | 65.422 | 6.22E−15 | 2.11E−12 | EPSTI1 | epithelial stromal interaction 1 |
| 2.2083 | 1.727 | 4.2706 | 121.15 | 4.93E−27 | 2.62E−24 | OASL | 2′-5′-oligoadenylate synthetase like |
| 2.1724 | 1.6504 | 4.8791 | 188.59 | 1.12E−41 | 7.41E−39 | IFIH1 | interferon induced with helicase C domain 1 |
| 2.1156 | 1.6829 | 6.6982 | 376.9 | 1.44E−82 | 1.76E−79 | STAT1 | signal transducer and activator of transcription 1 |
| 2.04 | 1.581 | 8.7036 | 427.94 | 1.19E−93 | 1.72E−90 | DTX3L | deltex E3 ubiquitin ligase 3L |
| 2.0071 | 1.6722 | 5.909 | 298.29 | 1.69E−65 | 1.68E−62 | PLSCR1 | phospholipid scramblase 1 |
| 2.0001 | 1.9709 | 4.1073 | 47.297 | 5.37E−11 | 1.45E−08 | ZNF625- | ZNF625-ZNF20 readthrough (NMD candidate) |
| | | | | | | ZNF20 | |
| 1.905 | 1.6937 | 3.5908 | 65.531 | 5.89E−15 | 2.04E−12 | IRF9 | interferon regulatory factor 9 |
| 1.9005 | 1.6755 | 2.7615 | 22.49 | 1.31E−05 | 0.0013267 | GBP1 | guanylate binding protein 1 |
| 1.8816 | 1.2956 | 9.0956 | 296.13 | 4.96E−65 | 4.65E−62 | IFIT5 | interferon induced protein with |
| | | | | | | | tetratricopeptide repeats 5 |
| 1.8438 | 2.6727 | 2.1521 | 23.024 | 1.00E−05 | 0.0010654 | GRIP2 | glutamate receptor interacting protein 2 |
| 1.8249 | 1.3301 | 1.9432 | 14.442 | 0.00073102 | 0.036745 | PLSCR2 | phospholipid scramblase 2 |
| 1.8017 | 1.4185 | 7.6401 | 317.26 | 1.28E−69 | 1.36E−66 | SAMD9 | sterile alpha motif domain containing 9 |
| 1.7724 | 1.3604 | 5.3408 | 187.27 | 2.16E−41 | 1.38E−38 | HERC6 | HECT and RLD domain containing E3 |
| | | | | | | | ubiquitin protein ligase family member 6 |
| 1.7705 | 1.3244 | 4.4859 | 71.418 | 3.10E−16 | 1.24E−13 | UBE2L6 | ubiquitin conjugating enzyme E2 L6 |
| 1.7036 | 1.3829 | 6.7467 | 265.36 | 2.38E−58 | 2.11E−55 | PARP9 | poly(ADP-ribose) polymerase family member 9 |
| 1.6596 | 1.0636 | 4.7397 | 119.26 | 1.27E−26 | 6.53E−24 | TRIM22 | tripartite motif containing 22 |
| 1.6537 | 1.2988 | 4.069 | 58.028 | 2.51E−13 | 7.99E−11 | SP110 | SP110 nuclear body protein |
| 1.6507 | −0.1502 | 2.5445 | 37.187 | 8.41E−09 | 1.86E−06 | MYLK4 | myosin light chain kinase family member 4 |
| 1.6461 | 1.1795 | 3.2144 | 35.346 | 2.11E−08 | 4.26E−06 | UBA7 | ubiquitin like modifier activating enzyme 7 |
| 1.6313 | 1.5866 | 4.433 | 89.156 | 4.37E−20 | 2.11E−17 | ARL14EPP1 | ARL14EP pseudogene 1 |
| 1.6292 | 1.2168 | 4.7705 | 106.56 | 7.26E−24 | 3.61E−21 | PARP12 | poly(ADP-ribose) polymerase family member 12 |
| 1.5802 | 1.6322 | 2.9138 | 26.263 | 1.98E−06 | 0.00026104 | RAB1AP1 | RAB1A pseudogene 1 |
| 1.5745 | 1.12 | 3.1564 | 29.581 | 3.77E−07 | 6.07E−05 | IFI27 | interferon alpha inducible protein 27 |
| 1.5516 | 1.6406 | 2.8207 | 27.474 | 1.08E−06 | 0.00015811 | CYP2J2 | cytochrome P450 family 2 subfamily J member 2 |
| 1.5515 | 0.97201 | 3.5967 | 32.437 | 9.05E−08 | 1.70E−05 | SYT5 | synaptotagmin 5 |
| 1.5146 | 1.5784 | 2.4415 | 19.426 | 6.05E−05 | 0.0048674 | REC8 | REC8 meiotic recombination protein |
| 1.4569 | 1.0543 | 7.1461 | 146.05 | 1.93E−32 | 1.14E−29 | USP18 | ubiquitin specific peptidase 18 |
| 1.4401 | 2.2006 | 5.1219 | 27.256 | 1.21E−06 | 0.00016817 | ELFN2 | extracellular leucine rich repeat and |
| | | | | | | | fibronectin type III domain containing 2 |
| 1.3902 | 0.82668 | 3.3307 | 18.779 | 8.36E−05 | 0.0064027 | ACE2 | angiotensin I converting enzyme 2 |
| 1.36 | 1.295 | 5.3129 | 44.627 | 2.04E−10 | 5.24E−08 | CXCL8 | C—X—C motif chemokine ligand 8 |
| 1.3078 | 1.1937 | 6.9007 | 37.632 | 6.74E−09 | 1.51E−06 | H2AC6 | H2A clustered histone 6 |
| 1.2603 | 0.48038 | 3.8104 | 31.406 | 1.51E−07 | 2.74E−05 | IFI16 | interferon gamma inducible protein 16 |
| 1.2502 | 0.87272 | 8.3571 | 174.23 | 1.47E−38 | 8.98E−36 | PARP14 | poly(ADP-ribose) polymerase family member 14 |
| 1.244 | 1.1707 | 3.3145 | 14.832 | 0.00060168 | 0.032064 | OR51E2 | olfactory receptor family 51 subfamily E member 2 |
| 1.1763 | 0.97392 | 8.696 | 200.82 | 2.47E−44 | 1.71E−41 | EIF2AK2 | eukaryotic translation initiation factor 2 |
| | | | | | | | alpha kinase 2 |
| 1.1332 | −0.45046 | 3.552 | 34.224 | 3.70E−08 | 7.28E−06 | FBXL15 | F-box and leucine rich repeat protein 15 |
| 1.0949 | 0.79509 | 4.6993 | 22.026 | 1.65E−05 | 0.0015981 | SLC6A6 | solute carrier family 6 member 6 |
| 1.0592 | 0.68795 | 4.8391 | 60.561 | 7.07E−14 | 2.35E−11 | DDX60L | DExD/H-box 60 like |
| 1.0586 | −3.9904 | 4.6686 | 66.5 | 3.63E−15 | 1.34E−12 | FKBPL | FKBP prolyl isomerase like |
| 1.0167 | 0.61158 | 3.7267 | 14.183 | 0.00083222 | 0.040552 | IFI35 | interferon induced protein 35 |
| 0.8698 | 1.1839 | 3.6252 | 25.709 | 2.61E−06 | 0.00033872 | KREMEN2 | kringle containing transmembrane protein 2 |
| 0.85614 | 1.3047 | 3.7445 | 37.045 | 9.03E−09 | 1.97E−06 | INHBA | inhibin subunit beta A |
| 0.60454 | −1.2691 | 1.9918 | 14.734 | 0.0006317 | 0.03311 | RSPH10B2 | radial spoke head 10 homolog B2 |
| 0.40296 | 1.4471 | 2.4986 | 21.083 | 2.64E−05 | 0.002351 | FPR3 | formyl peptide receptor 3 |
| 0.32437 | 1.6498 | 1.6979 | 14.74 | 0.00062979 | 0.03311 | PDE2A | phosphodiesterase 2A |
| 0.2971 | −1.3841 | 4.5746 | 24.477 | 4.84E−06 | 0.00056699 | CXCR5 | C—X—C motif chemokine receptor 5 |
| 0.0016387 | −1.035 | 3.9289 | 28.453 | 6.63E−07 | 9.96E−05 | RIPPLY3 | ripply transcriptional repressor 3 |
| −0.151 | −1.6464 | 2.5644 | 23.457 | 8.06E−06 | 0.00088573 | TMPRSS13 | transmembrane serine protease 13 |
| −0.29246 | −1.5043 | 2.9939 | 27.77 | 9.33E−07 | 0.00013766 | HSPA12B | heat shock protein family A (Hsp70) member 12B |
| −0.39018 | −3.2416 | 0.8389 | 14.735 | 0.00063135 | 0.03311 | OR2W6P | olfactory receptor family 2 subfamily W |
| | | | | | | | member 6 pseudogene |
| −0.9212 | 1.0481 | 2.4626 | 19.105 | 7.10E−05 | 0.005575 | LHX6 | LIM homeobox 6 |
| −1.1054 | −0.18711 | 3.2197 | 16.383 | 0.00027703 | 0.017176 | CLDN2 | claudin 2 |
| −1.1267 | −1.4016 | 9.0029 | 27.408 | 1.12E−06 | 0.00015905 | ANKRD30BL | ankyrin repeat domain 30B like |
| −1.1349 | 0.20722 | 2.518 | 14.474 | 0.00071936 | 0.036273 | LMO1 | LIM domain only 1 |
| −1.1446 | −0.94771 | 3.9669 | 15.525 | 0.00042541 | 0.024037 | SRGAP2D | SLIT-ROBO Rho GTPase activating protein 2D |
| | | | | | | | (pseudogene) |
| −1.1891 | −0.0015398 | 4.5206 | 26.38 | 1.87E−06 | 0.0002503 | SPECC1L- | SPECC1L-ADORA2A readthrough |
| | | | | | | ADORA2A | (NMD candidate) |
| −1.2704 | −1.8716 | 4.1256 | 23.855 | 6.61E−06 | 0.00073607 | FMC1- | FMC1-LUC7L2 readthrough |
| | | | | | | LUC7L2 | |
| −1.2848 | −0.88484 | 6.7341 | 88.673 | 5.56E−20 | 2.60E−17 | H3P6 | H3 histone pseudogene 6 |
| −1.3599 | −2.2103 | 1.006 | 13.752 | 0.0010321 | 0.047947 | ADAD2 | adenosine deaminase domain containing 2 |
| −1.3833 | 0.077091 | 2.9318 | 27.335 | 1.16E−06 | 0.00016354 | APOBEC3D | apolipoprotein B mRNA editing enzyme catalytic |
| | | | | | | | subunit 3D |
| −1.4217 | −1.0934 | 2.3448 | 18.119 | 0.0001163 | 0.0085002 | CYP26C1 | cytochrome P450 family 26 subfamily C member 1 |
| −1.5358 | −0.17689 | 3.15 | 24.833 | 4.05E−06 | 0.00048905 | TMEM272 | transmembrane protein 272 |
| −1.6351 | 1.1156 | 1.6829 | 16.453 | 0.00026747 | 0.016648 | IRX4 | iroquois homeobox 4 |
| −1.6583 | 0.058962 | 1.7474 | 14.75 | 0.00062658 | 0.03311 | CST7 | cystatin F |
| −1.7152 | 0.27984 | 3.449 | 29.492 | 3.94E−07 | 6.28E−05 | PCDHGC5 | “protocadherin gamma subfamily C, 5” |
| −1.7881 | −1.9206 | 1.4148 | 17.614 | 0.0001497 | 0.01034 | SERBP1P5 | SERPINE1 mRNA binding protein 1 pseudogene 5 |
| −2.0378 | −1.1984 | 3.0617 | 20.799 | 3.04E−05 | 0.0026221 | RPS28P7 | ribosomal protein S28 pseudogene 7 |
| −2.0497 | 0.1407 | 2.3942 | 25.451 | 2.97E−06 | 0.0003732 | MYRIP | myosin VIIA and Rab interacting protein |
| −2.1033 | −0.17562 | 3.7323 | 14.523 | 0.0007022 | 0.035633 | CCBE1 | collagen and calcium binding EGF domains 1 |
| −2.1153 | −0.8227 | 1.6676 | 17.81 | 0.00013569 | 0.0097389 | ANO4 | anoctamin 4 |
| −2.2277 | −1.4625 | 4.3468 | 27.063 | 1.33E−06 | 0.0001825 | RPL36A- | RPL36A-HNRNPH2 readthrough |
| | | | | | | HNRNPH2 | |
| −2.2726 | −1.7089 | 1.6856 | 16.46 | 0.00026653 | 0.016648 | EIF3FP3 | eukaryotic translation initiation factor 3 |
| | | | | | | | subunit F pseudogene 3 |
| −2.2829 | −1.1793 | 1.7004 | 21.808 | 1.84E−05 | 0.001733 | CLDN23 | claudin 23 |
| −3.8726 | −1.8859 | 1.7013 | 35.035 | 2.47E−08 | 4.92E−06 | FRG2C | FSHD region gene 2 family member C |

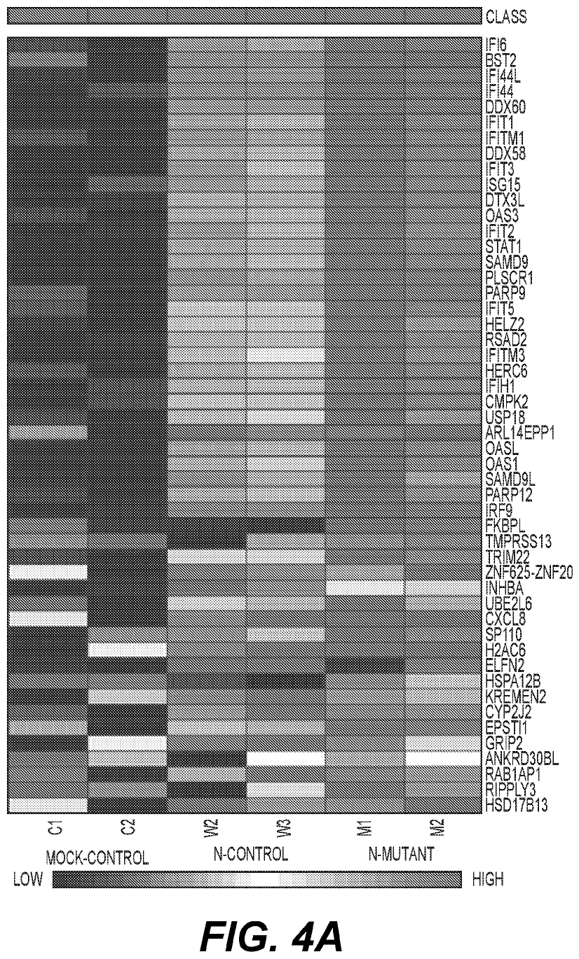

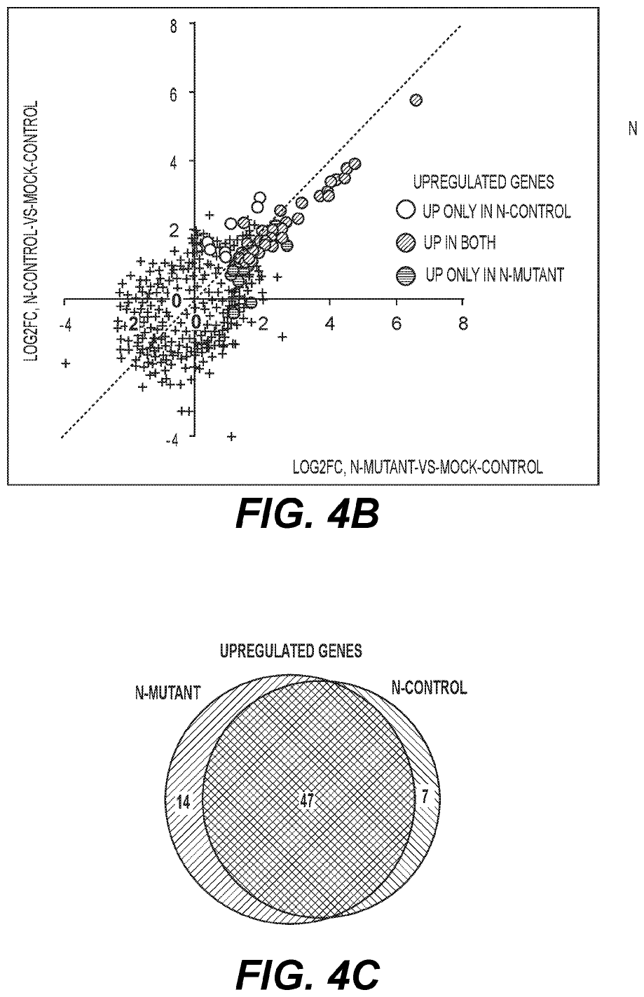

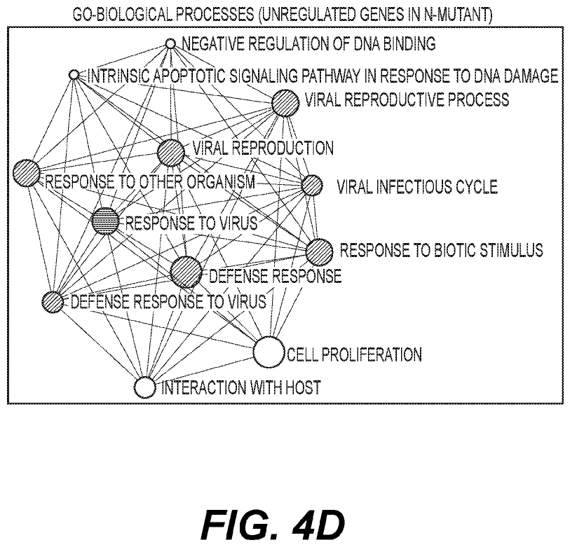

Among the DEGs, numerous interferons, cytokine, and immune-related genes are up-regulated, some of which are shown in FIG. 4 (see also, see Table 7). A robust overexpression of interferon-related genes in the N-mutant compared to N-control transfected cells (FIG. 4A-B) after adjusting for fold change was also seen (FIG. 7D). Also, we found overexpression of other genes such as ACE2, STAT1⁴⁴, and TMPRSS13⁴⁸ (FIG. 4A and Table 5) that are elevated in critical COVID-19 disease.

Pathway enrichment analysis of the uniquely up-regulated genes in the N-mutant condition (FIG. 4C) shows an overrepresentation of biological process pathways associated with response to the virus (FIG. 4D). Similarly, all up-regulated genes were related to substantially enriched pathways, such as interferon-related response, cytokine production, and viral reproductive processes (FIG. 7E). The enriched GO terms display an interconnected network highlighting the relationships between up-regulated overlapping genes sets in these pathways (FIGS. 4D and 7E). Taken together, these results suggest that the R203K/G204R mutations in the N protein may enhance its function in upregulating immune related genes; thus its expression in cells transfected to expression this mutant form of the N protein to confer a beneficial increased expression of immune related genes.

### DISCUSSION

From 892 samples collected across the country over the course of approximately 6 months we have analyzed the dynamics of transmission and diversity of SARS-CoV-2 in Saudi Arabia. The lineage analysis of assembled genomes highlights the repeated influx of SARS-CoV-2 lineages into the Kingdom. The earliest estimated importation dates point to an entry during the early stages of the pandemic, with the first importation likely to have an Asian origin. From estimates of viral genetic diversity and reproduction rate, we find that decreased diversity and reproduction rate coincides with imposed national curfews and is followed by an observed drop in reported COVID-19 cases.

The COVID-19 patient data studied here allowed detection of three SNPs—underlying the N protein R203K and G204R mutations —significantly associated with higher viral load. It is worth noting that two studies have found higher viral load has in infected patients to be associated with severity and mortality.

The data shows that the mutant N protein containing R203K and G204R changes has higher oligomerization and stronger viral RNA binding ability, suggesting a potential link of these mutations with efficient viral genome packaging. The R203K and G204R mutations are in close proximity to the recently reported RNA-mediated phase separation domain (aa 210-246)⁴² that is involved in viral RNA packaging through phase separation. This domain was thought to enhance phase-separation also through protein-protein interactions⁴². Further studies are needed to examine any definite link between KR mutation and phase-separation; however, the differential interaction of host proteins, as shown in our study could affect this process.

Moreover, the functional activities of the N protein at different stages of the viral life cycle are regulated by phosphorylation-dependent physiochemical changes in the LKR region⁴⁰. Although all individual phosphorylation sites may not be functionally important^{32,54}, the specific enhancement of phosphorylation at serine 206 in the mutant N protein shown in this study hints at its functional significance. The serine 206 can form a phosphorylation-dependent binding site for protein 14-3-3, involved in cell cycle regulatory pathways regulating human and virus protein expression⁵⁵. Multiple lines of evidence show that N protein phosphorylation is critical for its dynamic localization and function at replication-transcription complexes (RTC), where it promotes viral RNA transcription and translation by recruiting cellular factors^{38-40,56-59} The enrichment of glycogen synthase kinase 3 A (GSK3A) with the mutant N protein, could specifically phosphorylate serine 206 in the R203K/G204R mutation background. GSK3 was shown to be a key regulator of SARS-CoV replication due to its ability to phosphorylate N protein 39. Phosphorylation of serine 206 acts as priming site for initiating a cascade of GSK-3 phosphorylation events^{39,40}. Also, GSK3 inhibition dramatically reduces the production of viral particles and the cytopathic effect in SARS-CoV-infected cells 39. Finally, our analysis of the transcriptome in transfected cells suggests that the mutant N protein induce overexpression of interferon-related genes that can aggravate the viral infection by inducing cytokine storm.

In conclusion, the results herein results highlight the major influence of the R203K/G204R mutations on the essential properties and phosphorylation status of SARS-CoV-2 N protein the heterologous expression of which led to increased expression of immune related genes in cells.

References

| | |
|---|---|
| 1 | Organization, W. H. Coronavirus disease (COVID-19) Weekly |
| | Epidemiological Update and Weekly Operational Update, (website: |
| | who.int/emergencies/diseases/novel-coronavirus-2019/situation-reports) |
| | (2020). |
| 2 | Dong, E. et al. Lancet Infect Dis 20, 533-534, doi: 10.1016/S1473- |
| | 3099(20)30120-1 (2020). |
| 3 | Center, J. H. U. M. C. R. COVID-19 Dashboard, (website: |
| | coronavirus.jhu.edu/map) (2020). |
| 4 | Ebrahim, et al. Lancet 395, e48, doi: 10.1016/S0140-6736(20)30466-9 |
| | (2020). |
| 5 | Memish, et al. Lancet Infect Dis, doi: 10.1016/S1473-3099(20)30425-4 |
| | (2020). |
| 6 | Tuite, et al. Ann Intern Med 172, 699-701, doi: 10.7326/M20-0696 (2020). |
| 7 | News, A. Saudi Arabia announces first case of coronavirus, (website: |
| | arabnews.com/node/1635781/saudi-arabia) (2020). |
| 8 | Gussow, et al. Proceedings of the National Academy of Sciences of the |
| | United States of America 117, 15193-15199, doi: 10.1073/pnas.2008176117 |
| | (2020). |
| 9 | Lu, et al. Cell 181, 997-1003 e1009, doi: 10.1016/j.cell.2020.04.023 (2020). |
| 10 | Elbe, et al. Global Challenges 1, 33-46, doi: 10.1002/gch2.1018 (2017). |
| 11 | Shu, et al. Euro Surveill 22, doi: 10.2807/1560-7917.ES.2017.22.13.30494 |
| | (2017). |
| 12 | Hadfield, et al. Bioinformatics 34, 4121-4123, (2018). |
| 13 | Volz, et al. Cell 184, 64-75 ell, doi: 10.1016/j.cell.2020.11.020 (2021). |
| 14 | Davies, et al. Nature, doi: 10.1038/s41586-021-03426-1 (2021). |
| 15 | Lin, et al. Cell Host Microbe 29, 489-502 e488, |
| | doi: 10.1016/j.chom.2021.01.015 (2021). |
| 16 | Volz, et al. Syst Biol 67, 719-728, doi: 10.1093/sysbio/syy007 (2018). |
| 17 | Leary, et al. bioRxiv, 2020.2004.2010.029454, |
| | doi: 10.1101/2020.04.10.029454 (2020). |
| 18 | Morel, et al. Molecular biology and evolution, doi: 10.1093/molbev/msaa314 |
| | (2020). |
| 19 | Turakhia, et al. PLoS genetics 16, e1009175, |
| | doi: 10.1371/journal.pgen.1009175 (2020). |
| 20 | The Lancet Microbe 1, e99-e100, doi: 10.1016/S2666-5247(20)30054-9 |
| | (2020). |
| 22 | Yi, et al. Infectious Diseases 71, 884-887, doi: 10.1093/cid/ciaa219 (2020). |
| 23 | Richard, et al. bioRxiv, 2020.2012.2015.422866, |
| | doi: 10.1101/2020.12.15.422866 (2020). |
| 24 | Wu, et al. J Med Virol, doi: 10.1002/jmv.26597 (2020). |
| 25 | Rambaut, et al. Nat Microbiol 5, 1403-1407, doi: 10.1038/s41564-020- |
| | 0770-5 (2020). |
| 26 | Jackson, B. et al. Recombinant SARS-CoV-2 genomes involving lineage |
| | B.1.1.7 in the UK, (website: virological.org/t/recombinant-sars-cov-2- |
| | genomes-involving-lineage-b-1-1-7-in-the-uk/658) (2021). |
| 28 | Korber, et al. Cell 182, 812-827 e819, doi: 10.1016/j.cell.2020.06.043 |
| | (2020). |
| 29 | Ng, P. C. & Henikoff, S. Nucleic Acids Res 31, 3812-3814, doi: |
| | 10.1093/nar/gkg509 (2003). |
| 30 | McBride, R., et al. Viruses 6, 2991-3018, doi: 10.3390/v6082991 (2014). |
| 31 | Rahman, M. S. et al. J Med Virol, doi: 10.1002/jmv.26626 (2020). |
| 32 | Guan, Q. et al. Int J Infect Dis 100, 216-223, doi: 10.1016/j.ijid.2020.08.052 |
| | (2020). |
| 33 | He, R. T. et al. Biochem Bioph Res Co 316, 476-483, doi: |
| | 10.1016/j.bbrc.2004.02.074 (2004). |
| 34 | Chang, et al. Plos One 8, doi: ARTN e6504510.1371/journal.pone.0065045 |
| | (2013). |
| 35 | Chao Wu, et al. BioRxiv, doi: 10.1101/2020.11.30.404905 (2020). |
| 36 | Gordon, D. E. et al. Nature 583, 459−+, doi: 10.1038/s41586-020-2286-9 |
| | (2020). |
| 37 | Lowrey, A. J., et al. Cell Commun Signal 15, doi: ARTN |
| | 2710.1186/s12964-017-0183-0 (2017). |
| 38 | Wu, C. H., et al. Cell Host Microbe 16, 462-472, doi: |
| | 10.1016/j.chom.2014.09.009 (2014). |
| 39 | Wu, C. H. et al. J Biol Chem 284, 5229-5239, doi: 10.1074/jbc.M805747200 |
| | (2009). |
| 40 | Carlson, C. R. et al. Mol Cell 80, 1092−+, doi: 10.1016/j.molcel.2020.11.025 |
| | (2020). |
| 41 | Savastano, A., et al. Nat Commun 11, doi: ARTN 604110.1038/s41467-020- |
| | 19843-1 (2020). |
| 42 | Lu, S. et al. Nature communications 12, 502, doi: 10.1038/s41467-020- |
| | 20768-y (2021). |
| 43 | Gill, S. E. et al. Intensive Care Med Exp 8, 75, doi: 10.1186/s40635-020- |
| | 00361-9 (2020). |
| 44 | Jain, R. et al. Comput Struct Biotechnol J 19, 153-160, |
| | doi: 10.1016/j.csbj.2020.12.016 (2021). |
| 45 | Nienhold, R. et al. Nature communications 11, 5086, doi: 10.1038/s41467- |
| | 020-18854-2 (2020). |
| 46 | Lieberman, N. A. P. et al. PLoS Biol 18, e3000849, |
| | doi: 10.1371/journal.pbio.3000849 (2020). |
| 47 | Sposito, B. et al. bioRxiv, 2021.2003.2030.437173, |
| | doi: 10.1101/2021.03.30.437173 (2021). |
| 48 | Kishimoto, M. et al. Viruses 13, doi: 10.3390/v13030384 (2021). |
| 49 | Fajnzylber, J. et al. Nature communications 11, 5493, doi: 10.1038/s41467- |
| | 020-19057-5 (2020). |
| 50 | Pujadas, E. et al. Lancet Respir Med 8, e70, doi: 10.1016/S2213- |
| | 2600(20)30354-4 (2020). |
| 51 | Chang, C. K., et al. Antiviral Res 103, 39-50, |
| | doi: 10.1016/j.antiviral.2013.12.009 (2014). |
| 52 | Lal, M. in Molecular Biology of the SARS-Coronavirus (ed Sunil K. Lal) |
| | 129-151 (2009). |
| 53 | Wegener, M. & Muller-McNicoll, M. Adv Exp Med Biol 1203, 83-112, |
| | doi: 10.1007/978-3-030-31434-7_3 (2019). |
| 54 | Bouhaddou, M. et al. Cell 182, 685-712 e619, |
| | doi: 10.1016/j.cell.2020.06.034 (2020). |
| 55 | Nathan, K. G. & Lal, S. K. Viruses 12, doi: 10.3390/v12040436 (2020). |
| 56 | Verheije, M. H. et al. J Virol 84, 11575-11579, doi: 10.1128/Jvi.00569-10 |
| | (2010). |
| 57 | Chen, H. Y. et al. J Virol 79, 1164-1179, doi: 10.1128/Jvi.79.2.1164- |
| | 1179.2005 (2005). |
| 58 | Peng, T. Y., et al. Febs J 275, 4152-4163, doi: 10.1111/j.1742- |
| | 4658.2008.06564.x (2008). |
| 59 | V'kovski, P. et al. Elife 8, doi: ARTN e4203710.7554/eLife.42037 (2019). |
| 60 | Bolger, A. M., et al. Bioinformatics 30, 2114-2120, |
| | doi: 10.1093/bioinformatics/btu170 (2014). |
| 61 | Li, H. & Durbin, R. Bioinformatics 26, 589-595, |
| | doi: 10.1093/bioinformatics/btp698 (2010). |
| 62 | McKenna, et al. Genome research 20, 1297-1303, |
| | doi: 10.1101/gr.107524.110 (2010). |
| 63 | Li, et al. Bioinformatics 27, 2987-2993, doi: 10.1093/bioinformatics/btr509 |
| | (2011). |
| 64 | Nguyen, et al. Molecular biology and evolution 32, 268-274, |
| | doi: 10.1093/molbev/msu300 (2015). |
| 65 | Sagulenko, et al. Virus Evol 4, vex042, doi: 10.1093/ve/vex042 (2018). |
| 66 | Tamura, et al. Molecular biology and evolution 10, 512-526, |
| | doi: 10.1093/oxfordjournals.molbev.a040023 (1993). |
| 67 | Hasegawa, et al. J Mol Evol 22, 160-174, doi: 10.1007/BF02101694 (1985). |
| 68 | Volz, et al. Virus Evolution 3, doi: 10.1093/ve/vex025 (2017). |
| 69 | Schliep, et al. Bioinformatics 27, 592-593, |
| | doi: 10.1093/bioinformatics/btq706 (2011). |
| 70 | Hu, et al. Sci China Life Sci 63, 706-711, doi: 10.1007/s11427-020-1661-4 |
| | (2020) |
| 71 | Li, et al. Bioinformatics 34, 3094-3100, doi: 10.1093/bioinformatics/bty191 |
| | (2018). |
| 73 | Zhang, et al. Sci Data 6, doi: ARTN 278 |
| 74 | Liu, et al. Epigenet Chromatin 12, doi: 10.1186/s13072-019-0322-5 (2019). |
| 75 | Cox, et al. Nat Biotechnol 26, 1367-1372, doi: 10.1038/nbt.1511 (2008). |
| 76 | Wu, et al. Nature 559, 637−+, doi: 10.1038/s41586-018-0350-5 (2018). |
| 77 | Shah, et al. J Proteome Res 19, 204-211, |
| | doi: 10.1021/acs.jproteome.9b00496 (2020). |
| 78 | Zeng, et al. Biochem Bioph Res Co 527, 618-623, |
| | doi: 10.1016/j.bbrc.2020.04.136 (2020). |
| 79 | Aken, B. L. et al. Database (Oxford) 2016, doi: 10.1093/database/baw093 |
| | (2016). |
| 80 | Yates, A. D. et al. Ensembl 2020. Nucleic acids research 48, D682-D688, |
| | doi: 10.1093/nar/gkz966 (2020). |
| 81 | Bray, N. L., et al. Nat Biotechnol 34, 525-527, doi: 10.1038/nbt.3519 |
| | (2016). |
| 82 | Zhou, G. et al. Nucleic acids research 47, W234-W241, |
| | doi: 10.1093/nar/gkz240 (2019). |

References

| | |
|---|---|
| 1 | Algaissi, A. A., et al. J Infect Public Health 13, 834-838, |
| | doi: 10.1016/j.jiph.2020.04.016 (2020). |
| 2 | Li, H. Bioinformatics 27, 2987-2993, doi: 10.1093/bioinformatics/btr509 |
| | (2011). |
| 3 | Korber, B. et al. Cell 182, 812-827 e819, doi: 10.1016/j.cell.2020.06.043 |
| | (2020). |
| 4 | Lorenzo-Redondo, R. et al. medRxiv, 2020.2005.2019.20107144, |
| | doi: 10.1101/2020.05.19.20107144 (2020). |
| 5 | Volz, E. et al. Cell 184, 64-75 e11, doi: 10.1016/j.cell.2020.11.020 (2021). |
| 6 | Barbu, M. G., et al. Front Med (Lausanne) 7, 567199, |
| | doi: 10.3389/fmed.2020.567199 (2020). |
| 7 | Reilev, M. et al. Int J Epidemiol 49, 1468-1481, doi: 10.1093/ije/dyaa140 |
| | (2020). |
| 8 | Yang, J. et al. Int J Infect Dis 94, 91-95, doi: 10.1016/j.ijid.2020.03.017 |
| | (2020). |
| 9 | Espinosa, O. A. et al. Rev Inst Med Trop Sao Paulo 62, e43, |
| | doi: 10.1590/S1678-9946202062043 (2020). |
| 10 | Zhou, F. et al. Lancet 395, 1054-1062, doi: 10.1016/S0140-6736(20)30566- |
| | 3 (2020). |
| 11 | Pantea Stoian, A. et al. Scientific reports 10, 21613, doi: 10.1038/s41598- |
| | 020-78575-w (2020). |
| 12 | Li, B. et al. Clin Res Cardiol 109, 531-538, doi:10.1007/s00392-020- |
| | 01626-9 (2020). |

1. A pharmaceutical composition comprising: (a) a polypeptide fragment of a SARS-CoV-2 N protein/peptide comprising a R203K/G204R mutation relative to SEQ ID NO:1 or (b) a nucleic acid, preferably, an mRNA encoding a SARS-CoV-2 N protein/peptide comprising an R203K/G204R mutation relative to SEQ ID NO:1.

2. The composition of claim 1, comprising one or more nanoparticles.

3. The composition of claim 1, wherein the nucleic acid is in an expression vector; optionally, wherein the nucleic acid further comprises a 5′ untranslated region (UTR) and a 3′ UTR, a poly(A) tail, and/or a 5′ cap analog.

4. The composition of claim 2, wherein the nanoparticle is a lipid nanoparticle optionally comprising an ionizable cationic lipid, a neutral lipid, a sterol, and a PEG-modified lipid.

5. The composition of claim 1, wherein the nucleic acid is mRNA.

6. The composition of claim 1, wherein the expression vector is selected from the group consisting of plasmid, minicircle DNA (mcDNA) and viral vector.

7. The composition of claim 6, wherein the vector is selected from the group consisting of bacteriophage, baculoviruses, tobacco mosaic virus, herpes virus, cytomegalo virus, retrovirus, vaccinia virus, adenovirus and adeno-associated virus.

8. The composition of claim 1, comprising an mRNA encoded by SEQ ID NO: 38.

9. The composition of claim 1, comprising a peptide selected from the group consisting of SEQ ID Nos. 34, 35, 36 and 39.

10. The composition of claim 1, further comprising an adjuvant.

11. The composition of claim 1, comprising mRNA encoded by a nucleic acid molecule up to 50%, 60%, 70%, 80%, 90%, 91%, 92%, 93%, 94%, 95%, 96%, 97%, 98%, or 99% identical to SEQ ID NO:38.

12. A method of eliciting an immune response in a subject comprising administering to the subject the composition of claim 1.

13. The method of claim 12, wherein the immune response comprises antibody production.

14. The method of claim 12, wherein the immune response comprises upregulation of immune related genes.

15. The method of claim 12, wherein the immune response is an anti-viral immune response.

16. The method of claim 12, wherein the composition increases expression of at least one immune related gene selected from the group consisting of OAS1 (2′-5′-oligoadenylate synthetase 1); OAS3 (2′-5′-oligoadenylate synthetase 3); BST2 (bone marrow stromal cell antigen 2); DDX60 (DExD/H-box helicase 60); DDX58 (DExD/H-box helicase 58); RSAD2 (radical S-adenosyl methionine domain containing 2); EIF2AK2 (eukaryotic translation initiation factor 2 alpha kinase 2); TRIM14 (Tripartite motif-containing 14); TRIM22 (tripartite motif containing 22); MX1 (MX Dynamin Like GTPase 1); and SHFL (shiftless Antiviral Inhibitor Of Ribosomal Frameshifting).

17. The method of claim 12, wherein the immune response is immune response against a coronavirus infection.

18. The method of claim 12, wherein the coronavirus is SARS-CoV-2.
